(19) United States
(12) Patent Application Publication
(10) Pub. No.: US 2003/0017976 A1
(43) Pub. Date: Jan. 23, 2003

(54) Therapeutic properties of liposome-encapsulated immunomodulators

(76) Inventors: Lynn E. Spitler, Tiburon, CA (US); Isaiah Fidler, Houston, TX (US)

Correspondence Address: Kate H. Murashige, Morrison & Foerster LLP, Suite 500, 3811 Valley Centre Drive, San Diego, CA, 92130-2332, US

(21) Appl. No.: 09/764,546

(22) Filed: Jan. 17, 2001

Related U.S. Application Data
(63) Continuation of application No. 09/226,075, filed on Jan. 6, 1999, now abandoned.
(60) Provisional application No. 60/070,717, filed on Jan. 7, 1998.

(51) Int. Cl. A61K 9/127; A61K 38/17

(52) U.S. Cl. 514/12; 424/450

(57) ABSTRACT

The present invention relates to the use of novel compositions of lipopeptides that are immunomodulators encapsulated as liposomes or free-form for the treatment of neoplasia and in reducing chemotherapeutically induced cellular pathology, including mucositis. These lipopeptides may be administered alone or in combination with a second antineoplastic agent.

### BACKGROUND OF THE INVENTION

1. Technical Field of the Invention

The present invention in the field of medicine and molecular biology relates to the use of novel compositions of immunomodulators as liposome encapsulated or free-form for treating neoplasia and in reducing chemotherapeutically induced cellular pathology, including mucositis.

2. Background Art

The immunomodulating properties of synthetic macrophage activators, such as muramyl tripeptide phosphatidylethanolamine (MTP-PE) or lipopeptides (CGP 31362) encapsulated into multilamellar liposomes (MLV) have been reported. Studies from several laboratories have demonstrated that systemic tumoricidal activation of macrophages by either intravenous or oral administration enhanced host defenses against infections and cancer, including the eradication of metastatic disease in murine tumor models (Fidler, I. J. et al., Proc Natl Acad Sci, USA (1981) 78:1680-1684; Fidler, I. J., Cancer Immunol and Immunother (1986) 21:169-173; Dinney, C. P. N. et al., Cancer Res (1991) 51:3741-3747; Dinney, C. P. N. et al., Cancer Res (1992) 52:1155-1161) and canine osteosarcoma (MacEwen, E. G. et al., J Nat Canc Inst (1989) 81:935-938). MTP-PE was rigorously investigated in Phase I and II clinical trials (Murray, J. L. et al, J Clin Oncol (1989) 7:1915-1925; Kleinerman, E. S. et al., Cancer Res (1989) 49:4665-4670) which showed that systemic administration of MLV-MTP-PE caused localization of the MLV to the liver, lungs, lymph nodes and spleens of cancer patients. These studies were extended to further clinical evaluation in recurrent osteosarcoma (Kleinerman, E. S. et al., J Clin Oncol (1992) 10:1310-1316; Kleinerman, E. S. et al., Canc Immunol and Immunother (1992) 34:211-220).

Applicant has reported that systemic administration of MTP-PE can be combined with myelosuppressive therapy, such as doxorubicin (DXR), cisplatin, irradiation and ifosfamide, with no additional toxicity (Killion, J. J. et al., Oncol Res (1992) 4:413-418); indeed, administration of either free-form MTP-PE or liposome-encapsulated MTP-PE prevented the monocytopenia normally associated with these treatment modalities (Killion, J. J. et al., Oncol Res (1994) 6:357-364). These findings on the restorative properties of macrophage activators motivated experiments designed to maintain the structural integrity of intestinal epithelium and the protection of mucosal leukocytes during chemotherapy of mice given oral feedings of MTP-PE (Killion, J. J. et al., Canc Biother and Radiopharmaceut (1996) 11:363-371).

The cellular and molecular basis of these biological effects differs between MTP-PE and MLV-CGP 31362, in part because of the interaction of different signaling pathways toward cellular activation (Fidler, I. J. et al., Lymphokine Res (1990) 9:449-463; Utsugi, T. et al., Canc Immunol Immunother (1991) 33:285-292; Dong, Z. et al., J Leukocyte Biol (1993) 53:53-60; Dong, Z. et al., J Exper Med (1993) 177:1071-1077). Lymphocyte populations are also involved in mediating the antitumor (and probably tissue-protecting) effects of these immunomodulators (Killion, J. J. et al., Canc Biother and Radiopharmaceut (1996) 11:363-371; Utsugi, T. et al., Canc Immunol and Immunother (1991) 33:375-381). Activation of macrophages can result in the synthesis and release of numerous cytokines with a myriad of local and systemic effects (Nathan, C. F. J Clin Invest (1987) 79:319-326).

Advances in the therapeutic properties of macrophage-mediated immunomodulation can be obtained by the design of new activating molecules that have defined properties. These compounds include salts of aminosulfonic acid derivatives (Baschang et al; Aminosulfonic acid derivatives and processes for their preparation, U.S. Pat. No. 5,342,977; issued Aug. 30, 1994; which is hereby incorporated by reference in its entirety herein). Applicant has conducted a series of preliminary studies using one of these compounds designated JBT3002, designed to characterize the cellular parameters of tumoricidal activation. In addition, Applicant has recognized the potential use of this lipopeptide in the prevention of gut tissue damage due to chemotherapy as well as its use with chemotherapy in therapy of metastatic colon carcinoma. The pluripotential use of muramyl tripeptide analogues (reviewed in Killion, J. J. et al., Immunomethods (1994) 4:273-279) are compounds that warrant evaluation as therapeutic candidates for study in new clinical applications.

### DISCLOSURE OF THE INVENTION

The present invention is drawn to the use of compositions comprising isolated ψ-amino-C1-C3AL-kanesulfonic acid lipopeptides represented by the general formula (FIG. 1) and more specifically directed to N-acylated derivatives of ψ-amino-C1-C3AL-kanesulfonic acid. (FIG. 2) One derivative, JBT3002, is a synthetic analogue of a fragment of lipopeptide from the outer wall of Gram negative bacteria. This highly lipophilic molecule is soluble in chloroform and thus can be inserted into the bilayer membranes of phospholipid liposomes. Herein JBT3002 is shown to be a potent activator of cytokine production and tumoricidal properties in human blood monocytes and agent that stimulates several intracellular signaling pathways in human monocytes that are also activated by LPS, i.e., induction of tyrosine phosphorylation of proteins with apparent mass of 38- and 42-kDa, activation of c-Jun NH₂-terminal kinase 1 (JNK1), and activation of extracellular signaling-regulated kinases (Erks). In contrast to LPS, activation of monocytes by JBT3002 is not dependent on serum and is not mediated by binding to CD14. Other lipopeptides contemplated for use in the claimed invention include but are not limited to MTP-PE and CGP31362.

This invention is further drawn to pharmaceutical compositions of lipopeptides comprising ψ-amino-C I-C3AL-kanesulfonic acid derivatives and methods of their use for the treatment of neoplasia in subjects. Such pharmaceutical compositions comprise a therapeutically effective amount of the lipopeptide and a pharmaceutically acceptable carrier. Such pharmaceutical compositions may further include the insertion of the lipopeptide directly into bilayer membranes of phospholipid multilamellar vesicles (MLV) liposomes. The lipopeptide not inserted into MLV liposomes is considered to exist in free-form.

Additionally, pharmaceutical compositions may include in a pharmaceutically acceptable carrier the lipopeptide as a single active agent or in combination with a therapeutically effective amount of a second anti-neoplastic agent. One embodiment contemplates that the pharmaceutical composition of a lipopeptide, further comprising a pharmaceutically acceptable carrier in tablet form. A preferred embodiment contemplates that the lipopeptide has the structure or formula as represented in FIG. 2. A still further preferred embodiment of this invention contemplates that the lipopeptide represented in FIG. 2 is JBT3002.

Another embodiment of the present invention provides for a method of upregulating IL-15 production by administering to a subject a pharmaceutical composition that comprises an isolated lipopeptide comprising the formula represented in FIG. 1. A preferred embodiment contemplates that this lipopeptide has the structure or formula as represented in FIG. 2. A still further preferred embodiment of this invention contemplates that the lipopeptide represented in FIG. 2 is JBT3002.

A still further embodiment of this invention contemplates a method of treating a subject being treated with a neoplastic agent or therapeutic in an amount sufficient to cause a side effect, which method comprises administering to said subject a pharmaceutical composition that in a therapeutically effective concentration upregulates IL-15 production. A preferred embodiment contemplates that this lipopeptide has the structure or formula as represented in FIG. 2. A further preferred embodiment of this invention contemplates that the lipopeptide represented in FIG. 2 is JBT3002.

This invention also relates to a method of treating neoplasia by administering to a subject with neoplasia by a clinically acceptable route of delivery a therapeutically effective amount of the pharmaceutical composition comprising the lipopeptide and a pharmaceutically acceptable carrier. Another method of treating neoplasia contemplated by this invention relates to administering to a subject with neoplasia by a clinically acceptable route of delivery a therapeutically effective amount of the pharmaceutical composition comprising a first anti-neoplastic agent comprising a therapeutically effective amount of the lipopeptide in a multilamellar liposome or free-form; a therapeutically effective amount of a second anti-neoplastic agent; and a pharmaceutically acceptable carrier.

Drugs that are useful as a second anti-neoplastic agent in combination with the lipopeptide, include without limiting the present invention: CPT-11; other topoisomerase I inhibitors; paclitaxel (Taxol( brand) (Bristol-Myers Squibb); taxotere; modified taxane analogs; cisplatin; doxorubicin (Adriamycin); and ifosfamide.

Another aspect of the present invention relates to pharmaceutical compositions and methods of use of the lipopeptide immunomodulator, in a liposome encapsulated form or free-form, presented in combination with one or more cytokines in a pharmaceutically acceptable carrier. Cytokines contemplated by the present invention, include, for example: tumor necrosis factor alpha (TNF-α); interleukin-1-beta (IL-1β); interleukin-6 (IL-6); granulocyte colony stimulating factor (G-CSF); granulocyte macrophage colony stimulating factor (GM-CSF).

A further contemplation of the present invention relates to pharmaceutical compositions and methods of use for the treatment of a side effect resulting from the treatment of a subject with neoplasia, which method of use comprises: a therapeutically effective amount of the lipopeptide in a multilamellar liposome or free-form and a pharmaceutically acceptable carrier. This invention also relates to a method of treating a subject being treated with a neoplastic agent or therapeutic in an amount sufficient to cause a side effect, which method comprises administering to said subject a pharmaceutical composition comprising the lipopeptide in a multilamellar liposome or free-form and a pharmaceutically acceptable carrier, wherein the amount of the pharmaceutical composition is effective to alleviate or prevent said side effect. The side effects to a subject resulting from therapy with an anti-neoplastic agent include, but are not limited to: myelosupression, mucositis, and peripheral neuropathy, where the method comprises administering to said subject, in an amount effective to alleviate or prevent said side effect, the pharmaceutical composition containing the lipopeptide in a multilamellar liposome or free-form and a pharmaceutically acceptable carrier.

### BRIEF DESCRIPTION OF THE DRAWINGS

FIG. 1 shows the structural formula of ψ-amino-C1-C3AL-kanesulfonic acid lipopeptides.

FIG. 2 shows the structural formula of JBT3002.

FIG. 3 shows the binding and phagocytosis of multilamellar liposomes by mouse macrophages. PC or PC/PS liposomes (7:3 molar ratio) containing HBSS (control) or JBT3002 (0.1 mg/300 uM phospholipid) were incubated with adherent mouse macrophages for the indicated times at 37° C. in medium containing 10 U/ml IFN-γ. The values are the mean ±SD (Standard Deviation) of triplicate samples. This is one representative experiment of three.

FIG. 4 shows the time course of macrophage activation by liposomes-JBT3002. Macrophages (1×10⁵/well) were incubated for the indicated times with 50 nmol of liposomes containing 0.1 mg JBT3002/300 uM phospholipid. NO (nitrite) was determined at the indicated times. Cytotoxicity of K-1735 M2 cells was determined 72 h after coincubation with the macrophages. The values are the mean ±SD of triplicate cultures. This is one representative experiment of three.

FIG. 5 shows kinetics of protein-tyrosine phosphorylation induced by MLV-JBT3002. (A) Macrophages were incubated in medium without IFN-γ (control) or with medium containing 50 nmol MLV containing 0.1 mg JBT3002/300 uM phospholipids for the indicated times. The cells were washed and lysed in lysis buffer. Whole cell lysates (20 μg/lane) were separated by SDS-PAGE, transferred to nitrocellulose, and probed with antiphosphotyrosine monoclonal antibody 4G10 (0.2 μg/ml). The immunoreactive bands were detected by incubating the blots with horseradish peroxidase conjugated F(ab′)₂ of goat antimouse immunoglobulin G (1:2000) and developed by an ECL system. (B) Macrophages were pretreated for 20 h with medium containing 10 U/ml IFN-γ before LPS (1 μg/ml) or liposome-JBT3002 (50 μmol/well) were added for the indicated times. Western blot analysis was accomplished as described above.

FIG. 6 shows inhibition of macrophage activation by specific PTK inhibitors. Murine macrophages (1×10⁵) were incubated for 20 h in medium containing 25 nmol/38-mm² well of liposome-JBT3002 (0.1 mg/300 μM phospholipid) in the presence of genistein (A,B) or tyrphostin (C,D). The cultures were thoroughly washed and 1×10⁴ [³H]TdR-labeled cells were added. NO production (∘) was determined one day later and cytotoxicity (●) was determined 3 days later. The data are mean ±SD of triplicate cultures. This is one representative experiment of three.

FIG. 7 shows production of cytokines by MLV-JT3002-activated macrophages. PEM (1×10⁵/38-mm² well) were incubated for 24 hours with different concentrations of MLV-JT3002 (0.1 mg/300 μmol lipid) in the absence (□) or presence (▪) of 10 U/ml IFN-γ. The culture supernatants were assayed for nitrite content (A) using Griess reagent and for TNF-α (B) and IL-6 (D) by ELISA. IL-1α (C) was measured by ELISA of macrophage lysates. The data are the mean ±SD of duplicate cultures from one representative experiment of three. *P<0.01 and #P<0.05, compared with untreated macrophages.

FIG. 8 shows kinetics of cytokine production by MLV-JT3002-activated macrophages. PEM (1×10⁵/38-mm² well) were incubated for different times with 50 nmol/well of MLV-JT3002 (0.1 mg/300 μmol lipid). Culture supernatants were assayed for nitrite content (A) using Griess reagent and for TNF-α (B) and IL-6 (D) by ELISA. IL-1α (C) was measured by ELISA of macrophage lysates. The data are the mean ±SD of duplicate cultures from one representative experiment of three. *P<0.01 and #P<0.05, compared with untreated macrophages.

FIG. 9 shows northern blot analysis of cytokine mRNA induction of JT3002. PEM (5×10⁷/150 mm plates) were incubated for 4 hours in medium alone (lane 1), 10 U/ml IFN-γ (lanes 2, 4, 6, and 7), 100 ng/ml LPS (lanes 3 and 4), 5 μmol MLV-JT3002 (lanes 5 and 6), or 5 μmol/ml MLV-HBSS (lane 7). mRNA was extracted and analyzed by northern blotting using corresponding specific probes.

FIG. 10 shows activation of PEM by JT3002 is serum-independent. PEM (1×10⁵/38-mm² well) were incubated for 24 hours with LPS (100 μg/ml) or MLV-JT3002 (50 nmol/well, 0.1 mg/300 μmol lipid) with or without IFN-γ (10 U/ml) in serum-free EMEM or EMEM supplemented with 5% FBS. The culture supernatants were assayed for nitrite (A), TNF-α (B), and IL-6 (D), and the macrophage lysates were assayed for IL-1α (C). The data are the mean ±SD of duplicate cultures from one representative experiment of three. *P<0.01, compared with untreated macrophages.

FIG. 11 shows effects of protein kinase inhibitors on PEM activation by LPS or JT3002. PEM (1×10⁵/38-mm² well) were pretreated with genistein (100 μM), PD-98059 (10 μM), calphostin-C (250 nM), or H-89 (2.5 EM). After 20 minutes, LPS (100 ng/ml) or MLV-JT3002 (50 nmol/well of 0.1 mg/300 μmol lipid) were added together with IFN-γ (10 U/ml). The culture supernatants were assayed for nitrite (A) and TNF-α (B). The data are the mean ±SD of duplicate cultures from one representative experiment of two. *P<0.01 and P<0.05, compared with control PEM.

FIG. 12 shows activation of monocyte-mediated tumor cytotoxicity by MLV-JT3002. Monocytes (1×10⁵/38-mm² well) in 96-well plates were treated for 20 h with various concentrations of MLV-JT3002 prepared by encapsulating different amounts of JT3002 in 300 μM phospholipids. The medium did or did not contain 10 U/ml IFN-γ, Monocytes cultured in medium, LPS (100 ng/ml), or LPS (0.1 μg/ml) plus IFN-γ (10 U/ml) served as negative and positive controls, respectively. The treated monocytes were washed and incubated for 72 h with [³H]TdR-labeled A375SM cells (10⁴/well). The data shown are the mean ±SD of triplicate cultures. This is one representative experiment of three. MLV-JT3002 (∘); MLV-JT3002 plus IFN-γ (●).

FIG. 13 shows induction of cytokine production in monocytes by MLV-JT3002. Monocytes (1×10⁵/38-mm² well) in 96-well plates were incubated for 24 h with 100 nmol/well of MLV-JT3002 containing various concentrations of MLV-JT3002 (μg/300 μmol lipids). Cytokines in the culture supernatants were measured by ELISA. The data shown are the mean ±SD of triplicate cultures. This is one representative experiment of four. MLV-JT3002 (∘); MLV-JT3002 plus IFN-γ (●).

FIG. 14 shows production of TNF-α by monocytes exposed to MLV-JT3002, free-form JT3002, and LPS. (A) Monocytes (1×10⁵/38-mm² well) in 96-well plates were incubated for the indicated time periods with free-form JT3002 (1 ng/ml) or MLV-JT3002 (100 μmol/well, 1 mg JT3002/300 μmol lipids). TNF-α in the culture supernatants was determined by an ELISA kit. The data shown are the mean ±SD of triplicate cultures from one representative experiment of three. JT3002 (∘); MLV-JT3002 (●). (B) Monocytes were incubated for 24 h with various concentrations of LPS, free-form JT3002, or MLV-JT3002. The level of TNF-α in the culture supernatants was determined using an ELISA kit. The data are the mean ±SD of triplicate cultures. This is one representative experiment of three. JT3002 (●); MLV-JT3002 (▪); LPS (▴).

FIG. 15 shows serum-dependency for stimulation of cytokine production in monocytes exposed to LPS or MLV-JT3002. Monocytes (1×10^{5/38}-mm² well) in 96-well plates were incubated for 24 h with LPS (100 ng/ml) or MLV-JT3002 (100 nmol/well, 1 mg JT3002/300 μmol lipids) in serum-free EMEM (▪) or EMEM containing 5% FBS (□). The cytokines in the culture supernatants were measured using ELISA kits. The data shown are the mean ±SD of triplicate cultures. This is one representative experiment of three.

FIG. 16 shows inhibition of LPS-induced TNF-α production by anti-CD14 antibody. Monocytes (1×10⁵/38-mm² well) in 96-well plates were incubated for 24 h with medium alone (□) or with medium containing LPS (100 ng/ml) ( [symbol] ) or free-form JT3002 (1 ng/ml) (□) in the absence or presence of 80 μg/ml 3C10 monoclonal antibody (neat ascites). The level of TNF-α in the culture supernatants was measured using an ELISA kit. The data shown are the mean ±SD of triplicate cultures. This is one representative experiment of three.

FIG. 17 shows expression of cytokine mRNA. (A) Monocytes in 100-mm plates were incubated for 3 h in medium only (lane 1) or in medium containing 100 U/ml IFN-γ (lane 2), 100 ng/ml LPS (lane 3), IFN-γ (10 U/ml) plus LPS (0.1 μg/ml) (lane 4), 100 nmol/well MLV-HBSS (lane 5), IFN-γ (10 U/ml) plus 100 nmol/well MLV-HBSS (lane 6), 100 nmol/well MLV-fJT3002 (1 mg JT3002/300 μmol lipids) (lane 7), IFN-γ (10 U/ml) plus 100 nmol/well MLV-JT3002 (lane 8), 1 ng/ml free-form JT3002 (lane 9), or IFN-γ (10 U/ml) plus free-form JT3002 (1 ng/ml) (lane 10). Total cellular RNA was extracted and subjected to northern blot analysis as described in the Materials and Methods using corresponding cDNA probes. (B) Monocytes in 100-mm dishes were incubated for 3 h with medium only (lanes 1 and 4), or medium containing 100 ng/ml LPS (lanes 2 and 5), or 1 ng/ml JT3002 (lanes 3 and 6) in serum-free EMEM (lanes 1-3) or EMEM supplemented with 5% FBS (lanes 4-6). Total cellular RNA was extracted and subjected to northern blot analysis using human TNF-α or rat GAPDH cDNA probes. This is one representative experiment of three.

FIG. 18 shows western blot analysis of tyrosine phosphorylation, JNK1 band shift, and MAP kinase activation. Monocytes were incubated for 20 min with different concentrations of LPS or free-form JT3002. The cells were washed and lysed in a lysis buffer. Whole cell lysates (50 μg/lane) were separated by 10% SDS-PAGE, transferred to nitrocellulose, and probed with anti-phosphotyrosine monoclonal antibody 4G10 (0.2 μg/ml), anti-JNK1 monoclonal antibody 333.1 (1 μg/ml), or rabbit anti-activated MAP kinase antibody (0.1 μg/ml). The immunoreactive bands were detected or incubating the blots with horseradish peroxidase-conjugated goat anti-mouse or anti-rabbit immunoglobulin G (1:2000) and visualized by an ECL system. This is one representative experiment of three.

FIG. 19 shows serum-dependent and independent stimulation of intracellular signaling by LPS and JT3002. Monocytes were incubated for 20 min. with medium only (lanes 1 and 4), or in medium containing 100 ng/ml LPS (lanes 2 and 5), or 1 ng/ml free-form JT3002 (lanes 3 and 6) in serum-free medium (lanes 1-3) or in the presence of 5% FBS (lanes 4-6). Whole cell lysates (50 μg/lane) were analyzed by Western blotting as described in FIG. 8. This is one representative experiment of three.

FIG. 20 shows histological samples of intestinal villi and lumen demonstrating lack of GI toxicity in mice receiving MTP-PE prior to administration of CPT-11.

FIG. 21 shows histological samples of intestinal villi and lumen demonstrating lack of GI toxicity in mice receiving JBT3002 prior to administration of CPT-11.

FIG. 22 shows in the ileum that administration of CPT-11 alone produces disruption of the intestinal architecture (H&E).

FIG. 23 shows the response by macrophages and epithelial cells to JBT 3002 in upregulating IL-15 using the RT-PCR technique.

### MODES OF CARRYING OUT THE INVENTION

The demonstration that cells of the histiocyte-macrophage series can be activated by a variety of immunomodulatory agents and rendered cytotoxic against tumorigenic cells or virus-infected cells without affecting nontumorigenic or uninfected cells has prompted a search for ways to enhance the in vivo activation of monocytes-macrophages. In vivo activation of macrophages can occur by two major pathways: interaction with microorganisms and their products, e.g., endotoxins, or interaction with cytokines, e.g., interferon-gamma (IFN-γ), interleukin-1 (IL-1), tumor necrosis factor (TNF), macrophage colony stimulating factor, and monocyte chemotactic and activating factor. Efficient activation of macrophages to the tumoricidal state in situ can be accomplished by the encapsulation of hydrophilic or lipophilic immunomodulators within phospholipid liposomes. The systemic administration of multilamellar liposome vesicles (MLV) consisting of phosphatidylcholine (PC) and phosphatidylserine (PS) with encapsulated muramyl dipeptide or muramyl tripeptide phosphatidylethanolamine (MTP-PE) activates blood monocytes, lung macrophages, and liver macrophages. Repeated injections of these liposomes have been shown to enhance host resistance against viral infections, to eradicate established lung and liver metastases in murine tumor systems, to significantly prolong life and cures in dogs with autochthonous spontaneous lung metastases of osteogenic sarcoma, and to increase disease-free survival in children with chemotherapy-resistant osteogenic sarcoma lung metastases.

The interaction of macrophages with different immunomodulators can induce expression of different genes and hence, the production and release of different molecules. We have reported that liposomes containing a lipophilic analogue of Gram-negative bacteria cell wall (CGP31362) are potent activators of mouse macrophages and human blood monocytes. Monocytes incubated with this lipopeptide released interleukin-1 (IL-1), tumor necrosis factor (TNF), and prostaglandin E₂, whereas those incubated with liposomes containing MTP-PE released only TNF. Moreover, MLV containing CGP31362 produced superior activation of macrophages in situ and therapeutic effects on murine cancer metastases as compared to liposomes containing MTP-PE. These data suggested that this lipopeptide could be superior to MTP-PE. A major problem, however, with the use of CGP31362 has been low solubility even in organic solvents. We therefore tested a number of analogues of the lipopeptide with increased solubility to assess their entrapment in phospholipid MLV.

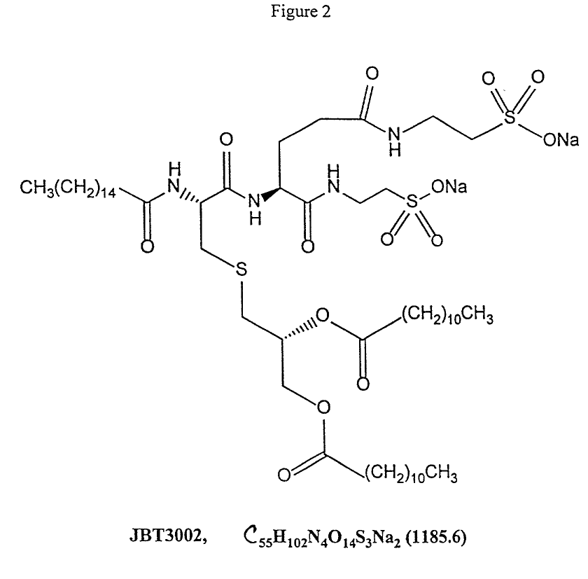

One such analogue, JBT3002, is a synthetic analogue of a fragment of lipopeptide from the outer wall of Gram-negative bacteria. This highly lipophilic molecule is derived from N-hexadecanol-S-[2(R)-3-diodecanoyloxypropyl]-L-cysteinyl-L-alanyl-D-isoglutaminyl-glycyl-taurine sodium salt. (FIG. 2) JBT3002 is soluble in chloroform and thus can be inserted directly into the bilayer membranes of phospholipid multilamellar vesicles (MLV) liposomes (in which form it is designated MLV-JBT3002).

We compared the efficiency of MLV-JBT3002 with that of MLV-CGP31362 and MLV-MTP-PE (CGP19835) for activating tumoricidal properties in mouse macrophages and determined the mechanism by which macrophages were rendered tumoricidal. Herein MLV-JBT3002 is shown to be a potent activator of tumoricidal properties in macrophages by mechanisms for tumoricidal activation and tumor cell lysis that differ from those associated with MLV-encapsulated muramyl peptide analogues, which depend on serum proteins and require intracellular signaling pathways.

This invention further envisions the administration of lipopeptides alone to patients or in combination with a these second antineoplastic agent. The administration of such lipopeptides is contemplated as a therapy to alleviate or prevent side effects arising from the treatment with a second antineoplastic agent. The side effects to a subject resulting from therapy with an anti-neoplastic agent include, but are not limited to: myelosupression, mucositis, and peripheral neuropathy. Such second antineoplastic agents include, but are not limited to: CPT-11; other topoisomerase I inhibitors; paclitaxel (Taxol® brand) (Bristol-Myers Squibb); taxotere; modified taxane analogs; cisplatin; doxorubicin (Adriamycin); and ifosfamide.

The use of these second antineoplastic agents is well known in the art. For example, U.S. Pat. No. 5,496,804, which is hereby incorporated by reference in its entirety herein, discloses various dosing regimens in the treatment of a patient using paclitaxel. Similarly, U.S. Pat. No. 5,565,478, which is hereby incorporated by reference in its entirety herein, discloses various dosing regimens in the treatment of a patient using paclitaxel.

The following examples are intended to illustrate but not to limit the invention.

### EXAMPLE 1

### MATERIALS AND METHODS

### Reagents

Eagle's minimum essential medium (EMEM), Hanks' balanced salt solution (HBSS), and fetal bovine serum (FBS) were purchased from M. A. Bioproducts (Walkersville, Md., U.S.A.). Recombinant mouse IFN-γ (specific activity 1×10⁵ U/mg protein) was obtained from Genentech (San Francisco, Calif., U.S.A.). Phenol-extracted Salmonella lipopolysaccharide (LPS) and N^{G}-monomethyl-L-arginine (NMA) were purchased from Sigma Chemical (St. Louis, Mo., U.S.A.). Genistein and tyrophostin were purchased from ICN Biomedicals (Costa Mesa, Calif., U.S.A.). The specific antiphosphotyrosine monoclonal antibody was purchased from UBI (Lake Placid, N.Y., U.S.A.), and JBT3002 was obtained from Jenner Technologies (San Raphael, Calif., U.S.A.). PC and PS were the gift of Novartis (Basel, Switzerland). All reagents, except for LPS, were free of endotoxin as determined by the Limulus Amoebocyte assay (detection limit 0.125 ng/ml) acquired from Associates of Cape Cod, Inc. (Walpole, Mass., U.S.A.).

### Animals

Specific pathogen-free, female C57BL/6 and 129/SVJ mice were purchased from Jackson Laboratory (Bar Harbor, Me., U.S.A.). Female C3H/HeN (LPS-responsive) and C3H/HeJ (LPS-nonresponsive) mice were purchased from the Animal Production Area, Frederick Cancer Research Facility (Frederick, Md., U.S.A.). iNOS gene knockout mice on the 129/SV background were the gift of Dr. Carl Nathan (Cornell University, New York, N.Y., U.S.A.) (19). Animals were maintained according to institutional guidelines in facilities approved by the American Association for Accreditation of Laboratory Animal Care and in accordance with current United States Department of Agriculture, Department of Health and Human Services, and the National Institutes of Health regulations and standards.

### Tumor Cells and Culture Conditions

K-1735 M2 melanoma cells syngeneic to C3H/HeN mice (Talmadge J E, et al. Nature (1982) 27:593-4), CT-26 colon carcinoma cells syngeneic to BALB/c mice (Dong Z, Radinsky R, Fan D, et al. J Natl Cancer Inst (1994) 86:913-20), and human A375-P melanoma cells (Kozlowski J M, et al. J Natl Cancer Inst (1984) 72:913-7) were used as target cells for in vitro mediated macrophage cytotoxicity assays. The K-1735 M2, CT-26, and A375-P cells were incubated in EMEM supplemented with sodium pyruvate, nonessential amino acids, 2 mM L-glutamine, and vitamin solution. For K-1735 M2 and CT-26 cells, the medium also contained 5% FBS, whereas for the A375-P cells, it contained 10% FBS. The cells were cultured in a humidified atmosphere at 37° C. and 5% CO₂ and air. All cell cultures were free of Mycoplasma, reovirus type 3, pneumonia virus of mice, K virus, encephalitis virus, lymphocyte choriomeningitis virus, ectromelia virus, and lactate dehydrogenase virus (assayed by M. A. Bioproducts).

### Preparation of Liposomes

PC (175 mg), PS (75 mg), CGP19835 (1 mg), and CGP31362 (0.125, 0.25, 0.5, or 1.0 mg) or JBT3002 (0.125, 0.25, 0.5, or 1.0 mg) were dissolved in chloroform under nitrogen. The clear solution was sterile-filtered through a Gelman-TF-200 (0.2-μm) filter. Aliquots (1 ml containing phospholipids with or without immunomodulators) were retroevaporated under a stream of nitrogen gas. The tubes with dry film were incubated overnight in a vacuum chamber at room temperature. Multilamellar liposomes were prepared by hydration of the lipid film with HBSS and high-speed agitation for 6 min. The liposomes were diluted into EMEM for addition to macrophage cultures.

### Isolation and Activation of Macrophages

Peritoneal exudate macrophages (PEM) were collected by peritoneal lavage from mice given an intraperitoneal injection of 2.0 ml of thioglycollate broth (Baltimore Biological Laboratory, Cockeysville, Md.) 4 days before harvest (Dong Z, O'Brian C A, et al. J Leukoc Biol (1993) 53:53-60; Xie K, Huang S, et al. Cancer Res (1995) 55:3123-31). The PEM were washed in Ca⁺²- an Mg²⁺-free HBSS and resuspended in serum-free medium: 1×10⁵ cells were plated into each 38-mm² well of 96-2311 culture plates (Falcon Plastics, Oxnard, Calif.). After a 90-min incubation, the nonadherent cells were removed by washing with medium. More than 98% of the adherent cell populations were macrophages according to morphology and phagocytic criteria (Saiki I, et al. J Immunol (1985) 135:684-8). These cultures were then fed with supplemented medium containing different combinations of activators of other reagents. After treatment, the cultures were washed and macrophage-mediated cytotoxicity against different tumor targets was determined.

### In Vitro Activation of Macrophages

Purified cultures of mouse macrophages were incubated at 37° C. for 18-24 h with 0.2 ml of control medium or with medium plus liposomes containing HBSS or immunomodulators. Liposomes were suspended in medium with or without rIFN-γ. After the incubation period, monocytes or macrophage cultures were thoroughly washed, and target cells were added as described below. Treatment of macrophages with MLV-HBSS or EMEM served as the negative control and treatment of macrophages with LPS and rIFN-γ served as the positive control (Saiki I, et al. J Immunol (1985) 135:684-8).

### Macrophage-mediated Cytotoxicity

Cytotoxicity was assayed by measuring release of radioactivity from target cell DNA as described previously (Dong Z, et al. J Exp Med (1993) 177:1071-7). Briefly, tumor target cells in their exponential growth phase were incubated in medium containing 0.25 μCi/ml of [³H]TdR (sp. Act. 2 Ci/mmol) (ICN Biomedicals, Inc., Irvine, Calif.) for 24 h. The cells were washed three times with HBSS to remove unbound radioactivity and then harvested by trypsinization (0.25% trypsin in 0.02% EDTA), washed, and resuspended in medium. Cells were plated (at 1×10⁴/well) into wells containing control or test macrophages to achieve an E/T cell ratio of 10:1. At this density, macrophages incubated in medium (control) were not cytotoxic to neoplastic cells (Weinstein SL, et al. Proc Natl Acad Sci USA (1991) 88:4148-52; Dong Z, et al. JLeukoc Biol (1993) 53:53-60). After 72 h of incubation, the cultures were washed three times with PGS, and adherent cells were lysed with 0.1 ml of 0.1 N NaOH. The lysate was harvested with harvester 96 (Tomtec, Orange, Conn.) and counted for residual radioactivity in a liquid scintillation counter. Macrophage-mediated cytotoxicity was calculated as follows:

Specific cytotoxicity (%)=[A−B]/A×100

where A=cpm in cultures of control macrophages and tumor cells and B=cpm in cultures of test (treated macrophages and tumor cells.

### Assay for Nitrite Production

Nitrite accumulation in the culture supernatant was measured in a colorimetric assay as described previously (Ding A E, et al. J Immunol (1988) 141:2407-12). At different times, 50-μl aliquots of supernatants were mixed with equal volumes of Griess reagent (1% sulfanilamide and 0.1% naphthylenediamine dihydrochloride in 2.5% phosphoric acid). The mixtures were incubated 10 min with shaking, and A540 was measured with the use of a microplate reader (Model 3550; Bio-Rad Corp., San Francisco, Calif.). The concentration of nitrite was determined by comparing it with a standard solution of sodium nitrite in medium.

### Phagocytosis of Liposomes

Macrophages (1×10⁵/38-mm² well) were plated in 96-well plates. MLV were prepared in the same manner as described above with 1% [¹²⁵I]phenylpropinoyl-PtdEtn. N-{3-(3-[¹²⁵I]iodo-4-hydroyxybenzyl)propionyl} dipalmitoyl-glycero-phospho-ethanolamine was prepared by using ¹²⁵I-labeled Bolton-Hunter reagent (spec. act. 2000 Ci/mmol) (New England Nuclear) as described earlier (Schroit A J, et al. Cancer Res (1982) 42:161-9). Adherent macrophages were incubated at 37° C. with either 25 or 50 nmol MLV. After different times, the monolayers were extensively washed with HBSS and the cells were lysed with 0.1 N NaOH. The lysate was absorbed on cotton, and radioactivity was monitored in a gamma counter (Utsugi T, et al. Cancer Immunol Immunother (1991) 33:285-92).

### Tyrosine Phosphorylation

Western blot analysis described previously (Dong Z, et al. J Exp Med (1993) 177:1071-7; Weinstein S L, et al. Proc Natl Acad Sci USA (1991) 88:4148-52; Dong Z, et al. J Leukoc Biol (1993) 53:53-60) was used to detect phosphorylation of tyrosine. Briefly, 1×10 ⁷ macrophages/60-mm dish were incubated with MLV-JBT3002 (test), LPS and IFN-γ (positive control), or EMEM alone (negative control). After different times, the cultures were washed five times with PBS containing 1 mM orthovanadate and 5 mM EDTA. The cells were harvested by scraping into a lysis buffer (IT Triton X-100, 20 mM Tris pH 8.0, 137 mM NaCl, 10% glycerol, 1 mM orthovanadate, 2 mM EDTA, 1 mM PHSF, 20 μM leupeptin, 0.15 U/ml aprotinin). The lysate was placed on ice for 20 min and then centrifuged at 14,000 g for 10 min at 4° C. The protein content of the supernatant was determined using the Lowry assay (BIORAD), and the concentrations were adjusted to 2 mg/ml protein using sample buffer. The samples were then boiled for 5 min, and 40 μg of protein was placed in a 10% SDS-PAGE gel and transferred onto nitrocellulose membranes with a pore size of 0.45 μm. The membranes were blocked with 3% bovine serum albumin and 1% ovalbumin in Tris buffered saline (TBS). The tyrosine-specific 4G10 monoclonal antibody was used as primary antibody (0.2 μg/ml diluted in TBS containing 0.1% Tween 20). The membranes were probed overnight at 4° C. and washed three times in Tween containing TBS. Immune complexes were detected by a goat-anti-mouse secondary antibody (Amersham Corp., Arlington Heights, Ill.) conjugated to horseradish peroxidase (1 h, 1:2000 dilution). The ECL system (Amersham) was used to develop the blotting filters.

### Statistical Analysis

All experimental results were analyzed for statistical significance by the use of the two-tailed Student” t-test.

### RESULTS

### Uptake of MLV by Macrophages

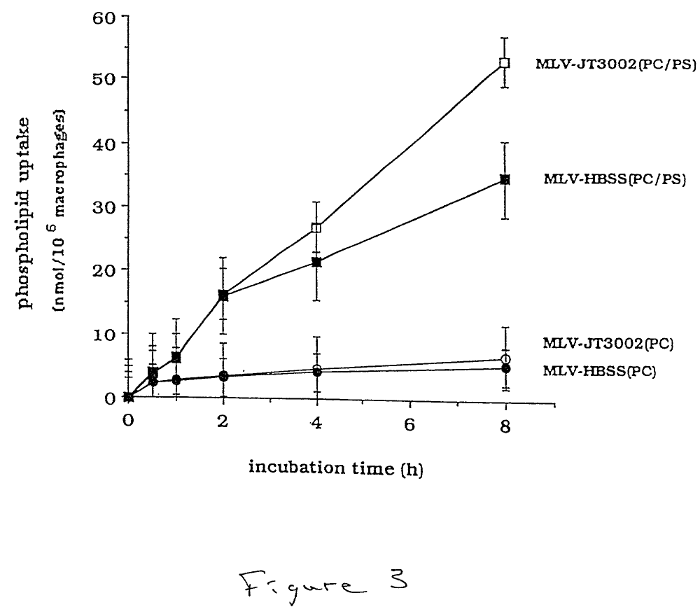

In the first set of experiments, the lipid composition of MLV is evaluated to determine its influence on the binding and phagocytosis by macrophages. MLV consisting of PC alone or PC/PS (7:3 molar ratio) containing 1 mg JBT3002/300 μM lipid were added to cultures of macrophages. The presence of PS in both the control MLV (containing HBSS) and MLV-JBT3002 (test) produced at least a 10-fold higher uptake than did PC; PC/PS MLV containing JBT3002 were taken up to a higher level than the HBSS control MLV (FIG. 3). These data closely agree with previous reports (Fidler I J, et al. Lymphokine Res (1990) 9:449-54; Utsugi T, et al. Cancer Immunol Immunother (1991) 33:285-92; Nii A, et al. J Immunother (1991) 10:236-46). MLV uptake was directly correlated with production of NO (data not shown). All subsequent studies were carried out with PC/PS MLV (7:3 molar ratio).

### Activation of Turnoricidal Properties in PEM

To rule out direct cytotoxicity effects, K-1735 M2 or A375P melanoma cells were incubated for 4 days with different concentrations of MLV-JBT3002 (0-25 nmol/38-mm² well, 1 mg JBT3002/300 μM phospholipids). Four days later, the viable tumor cells were counted. MLV-JBT3002 did not produce any direct cytotoxic effects (data not shown).

For all in vitro assays, PEM were incubated in medium in the presence or absence of 10 U/ml rIFN-γ. Negative controls consisted of PEM incubated with medium (endotoxin-free), whereas positive controls consisted of PEM incubated in medium containing 1 μg/ml LPS and 10 U/ml IFN-γ. PEM were also incubated with different concentrations of the following MLV preparations: MLV-MTP-PE (1 mg), MLV-CGP31362 (1 mg), MLV-JBT3002 (1 mg), and MLV-HBSS (control) (Table 1). After 24 h incubation, the culture supernatants were analyzed for NO production (nitrite/nitrate level), and the lysis of A375P cells was determined after 72 h of coincubation. PEM incubated in medium alone (data not shown) or in medium containing MLV-HBSS did not produce significant levels of NO or cytotoxicity. Macrophages treated with 50 nmol/well of MLV-MTP-PE were tumor-cytotoxic (19%, P<0.05). Macrophages treated with as little as 3 nmol MLV-CGP31362 or JBT3002 produced significant levels of NO (>20 μM, P<0.001) and tumor cytotoxicity (>60%, P<0.001).

In the next series of experiments, the concentration of JBT3002 was diluted in the MLV (0.1 mg, 0.02 mg, 0.004 mg, and 0.0008 mg/300 μM phospholipids) and PEM was incubated with different concentrations of MLV (containing the different amounts of JBT3002). The minimal concentration of JBT3002 required to generate significant levels of NO (20 μM) was calculated to be 0.12-0.15 ng available to 1×10⁵ PEM (Table 2). In parallel studies, the minimal concentration of JBT3002 was determined for significant activation of tumoricidal properties in PEM was 1.5 ng (available in MLV to 1×10⁵ cells).

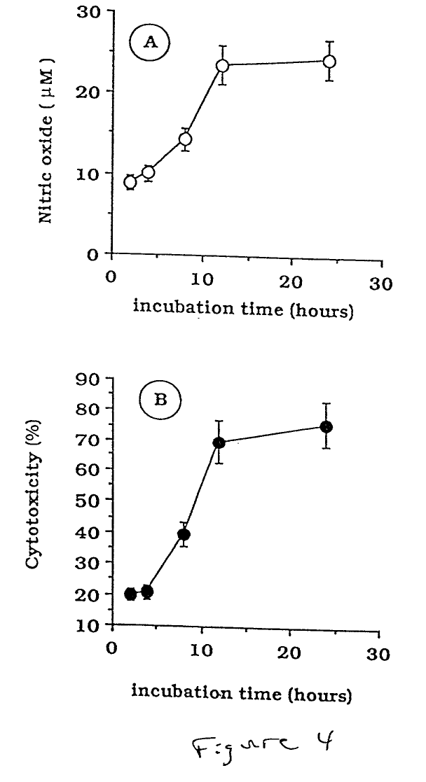

Next, the kinetics of PEM activation was determined for production of NO and tumor cell lysis (FIG. 4). Production of NO began within 2 h after incubation with PC/PS containing MLV-JBT3002. Significant levels of NO were produced after 12 h of incubation. The production of NO directly correlated with PEM-mediated cytotoxicity against K-1375 M2 cells (FIG. 4) or A375P cells (data not shown). These studies demonstrated that incubation of PEM with MLV containing 0.1-1.0 mg JBT3002/300 μM lipid can generate significant production of NO and tumor cytotoxicity.

### Mechanism of Macrophage-mediated Tumor Cytotoxicity

The production of NO by PEM activated in vitro with MLV JBT3002 was responsible for tumor cell toxicity. This conclusion is based on two experiments. In the first experiment, NMA was used as a specific inhibitor of iNOS (Xie K, et al. Cancer Res (1995) 55:3123-31). PEM were incubated with 50 umol/well of MLV containing 0.1 mg JBT3002/300 μM phospholipid. In the presence of 2 mM NMA, the PEM produced low levels of NO (1.6 μM) and no tumor cytotoxicity (1.3%). In the absence of NMA, the PEM produced 18.3 μM of NO and 50% tumor cytotoxicity (P<0.001).

In the second set of experiments, PEM was harvested from iNOS knockout mice (MacMiking J D, et al. Cell (1995) 81:641-50). The PEM were incubated in medium alone (negative control), medium containing only 10 U/ml IFN-γ, medium containing 10 U/ml IFN-γ and 1 μg/ml LPS (positive control), or medium containing different concentrations (0-50 nmol/well) of MLV containing 0.1 mg JBT3002/300 μM phospholipid. Production of NO was determined after 24 h of activation, and PEM-mediated cytotoxicity against CT-26 and K-1735 M2 cells was determined after 72 h of coincubation (Table 3). Treatment with LPS plus IFN-γ or MLV-JBT3002 induced high levels of NO production and tumor-mediated cytotoxicity (P<0.001) in PEM from normal 129/SJ mice (+/+). In PEM from heterozygous mice (+/−), LPS plus IFN-γ or MLV-JBT3002 decreased the production of NO and tumor cytotoxicity to about 50% of that in normal mice. Incubation of PEM from iNOS knockout mice (−/−) with LPS and IFN-γ or MLV containing JBT3002 did not induce production of NO nor significant cytotoxicity (Table 3).

### Activation of Macrophages from LPS-responsive and LPS-nonresponsive C3H Mice by MLV-JBT3002

To gain more insight into the activation mechanism of MLV-JBT3002, macrophages of LPS-responsive (C3H/HeN) and LPS-nonresponsive (C3H/HeJ) mice were used (Watson J, et al. J Immunol (1978) 120:422-5; Chedid L, et al. Infect Immunol (1976) 13:722-6). Macrophages were stimulated with 20, 2, 0.2, or 0.02 nmol/well MLV-JBT3002 in the presence or absence of 10 U/ml IFN-γ. Production of NO was determined after 24 h of incubation, and cytotoxicity against K-1735 M2 cells was determined after 72 h of PEM-tumor cell interaction (Table 4). PEM incubated in medium alone or medium containing only 10 U/ml IFN-γ did not produce NO or tumor cytotoxicity. LPS plus IFN-γ induced significant production of NO and cytotoxicity against K-1735 M2 cells in PEM from LPS-responsive C3H/HeN mice but not in PEM from LPS-nonresponsive C3H/HeJ mice (P<0.01).

In contrast to LPS plus IFN-γ, the incubation of PEM from C3H/HeN and C3H/HeJ mice with MLV-JBT3002 in medium containing 10 U/ml IFN-γ produced high levels of NO production (P<0.01) and tumor cytotoxicity (P<0.01), suggesting that the activation of PEM by LPS and MLV-JBT3002 may occur by different mechanisms.

### Duration of Tumoricidal Activity

To determine the duration of tumoricidal activation of MLV-JBT3002, PEM were incubated with 0.1 mg JBT3002/300 μM lipid, 0.1 nmol/38-mm² well. After 20 h, the PEM were thoroughly washed and refed with EMEM containing 5% FBS. Radioactively labeled tumor cells were added 1, 2, 3, or 4 days later, and tumor cytotoxicity was determined 72 h after PEM-tumor cell interaction (Table 5). Production of NO was also measured at different time points. Macrophages produced NO for at least 2 days after treatment with MLV-JBT3002. By day 4, this production decreased to nonsignificant levels. Similar results were obtained for tumor-mediated cytotoxicity.

We next determined whether the PEM could respond to a second treatment with MLV-JBT3002. PEM incubated with MLV-JBT3002 for 20 h were washed thoroughly, incubated for 4 days in medium containing 5% FBS, and then given another batch of MLV-JBT3002 (0.1 nmol/well). Both production of No (34 μM) and tumor cell cytotoxicity (41%) indicated that PEM can respond to a second challenge by MLV-JBT3002.

### Involvement of PTK in the Activation Mechanism

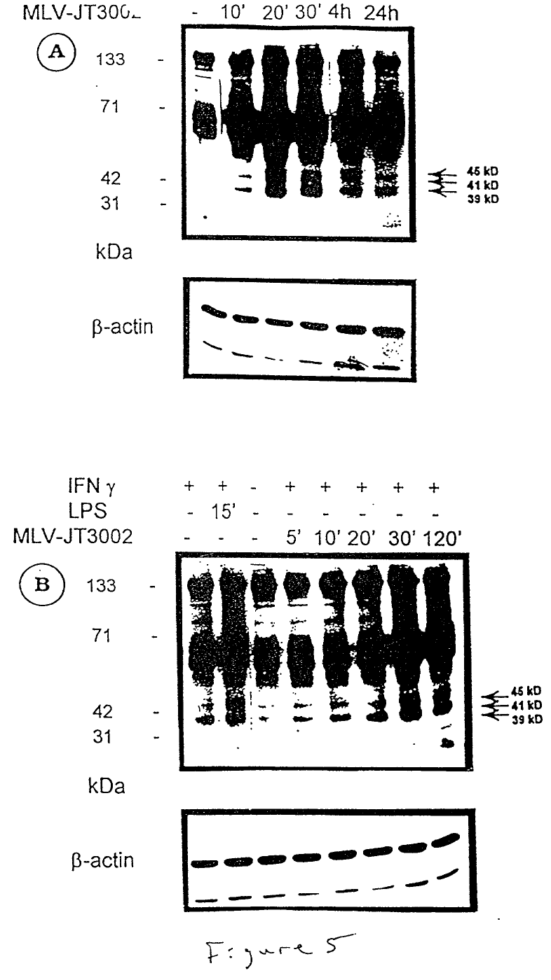

Since tumoricidal activation of murine macrophages by LPS or CGP31362 involves phosphorylation of PTK, whether the incubation of macrophages with MLV-JBT3002 also produced phosphorylation of protein tyrosine and whether inhibition of PTK activity would inhibit tumoricidal activation were determined. PEM were treated with MLV-JBT3002 for different times ranging from 10 min to 24 h. Cell lysates were analyzed for tyrosine phosphorylation using a specific antiphosphotyrosine monoclonal antibody (Dong Z, et al. J Immunol (1993) 151:2717-25; Weinstein S L, et al J Biol Chem (1992) 267:14955-63). A significant increase in phosphorylation of proteins with apparent molecule mass of 45, 41, and 39 kD (FIG. 5A) after 20 min. The phosphorylation was decreased 4 h later. Pretreatment of macrophages with IFN-γ did not alter the phosphorylation and its kinetics induced by MLV-JBT3002 (FIG. 5B). Maximal phosphorylation was observed 20-30 min after the addition of MLV-JBT3002. A similar pattern of tyrosine phosphorylation was observed in macrophages primed with IFN-γ and then triggered by LPS for 15 min (FIG. 5B).

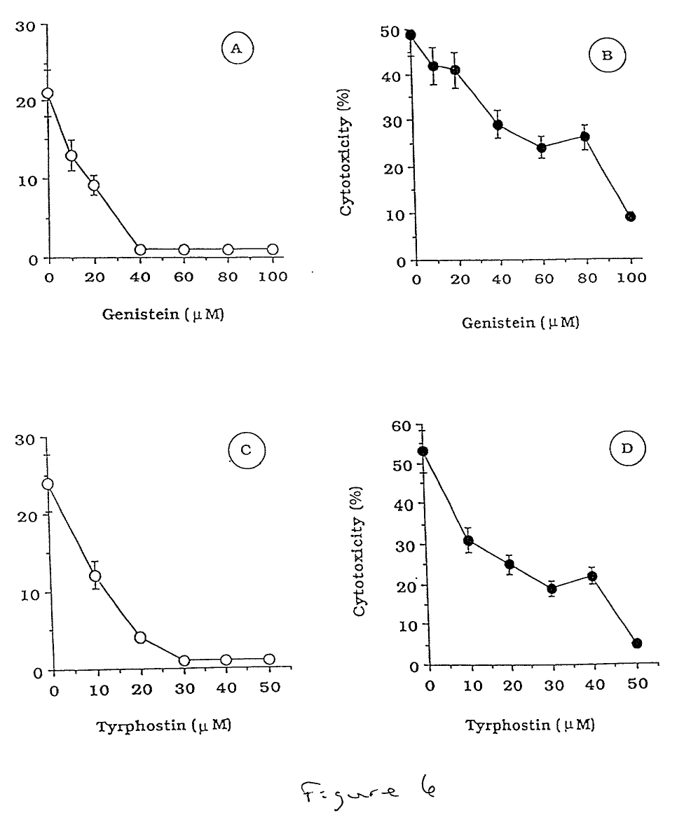

To determine whether PTK activity was essential for activation of PEM by JBT3002, the PEM were incubated with the immunomodulator in the presence of different concentrations of two PTK inhibitors, genistein and tyrophostin. Both genistein (FIG. 6A) and tyrophostin (FIG. 6B) inhibited production of NO and cytotoxicity of K-1735 M2 cells in a dose-dependent manner. Tyrophostin caused significant inhibition of tumor toxicity in a concentration range between 10 and 30 μM, whereas genistein required a minimal inhibition dose of 30-40 μM. Macrophage viability was not influenced by the inhibitors. The inhibitors did not produce direct antitumor activity (data not shown).

### Requirement of IFN-γ for Tumoricidal Activation

Efficient activation of macrophages by LPS requires priming by IFN-γ (Saiki I, et al. J Immunol (1985) 135:684-8). In the last set of studies, we incubated macrophages with 2.0 μg/ml LPS or 50 nmol/38-mm² well (1×10⁵ PEM) of 0.1 mg JBT3002/300 μM lipid in the absence or presence of different concentrations of IFN-γ (0-10 U/ml). Tumor cytotoxicity was measured 72 h after the addition of radiolabeled tumor cells to the activated macrophages. Induction of 50% tumor cell lysis was mediated by PEM activated with MLV-JBT3002 in the presence of 2 U/ml IFN-γ. Similar cytotoxicity by PEM activated by MLV-CGP31362 required 8 U/ml. PEM activated by LPS required 10 U/ml of IFN-γ.

### DISCUSSION

These results demonstrate that PC/PS (7:3 molar ratio) liposomes containing JBT3002, a synthetic lipopeptide derived from the outer wall of a gram-negative bacterium, are superior activators of NO production and tumoricidal properties in murine macrophages. This conclusion is based on the following results: (a) Liposomes containing JBT3002 produced tumoricidal activation of macrophages at significantly lower concentrations than liposomes containing MTP-PE or CGP31362. (b) Macrophage activation by MLV-JBT3002 required a lower concentration of IFN-γ (2 U/ml) than liposomes containing MTP-PE (10 U/ml). (c) Macrophages treated with low concentrations of MLV-JBT3002 produced significantly higher levels of NO than those treated with liposomes containing MTP-PE or CGP31362.

Following intravenous administration, >85% of MLV are cleared by phagocytic cells residing in the liver, spleen, lymph nodes, and bone marrow, and by circulating monocytes (4). This fate of circulating liposomes allows for specific targeting of encapsulated drugs, especially immunomodulating agents. The inclusion of negatively charged PS in PC liposomes has been shown to enhance their binding to and phagocytosis by macrophages (Schroit A J, et al Cancer Res (1982) 42:161-7). In agreement with these reports, herein is shown that at any time point, the phagocytosis of MLV consisting of PC and PS (7:3) was significantly higher than that of MLV consisting of only PC. Moreover, the uptake of PC/PS MLV containing JBT3002 was higher than that of PC/PS MLV containing saline (control), indicating that JBT3002 per se enhanced the uptake of MLV by macrophages. Whether this was due to rapid activation of cell surface reorganization or to the total negative charge of the MLV is unclear.

After interacting with cytokines or bacterial products, human and rodent monocytes-macrophages undergo activation, a process characterized by increased activity of protein tyrosine kinases (PTK), leading in turn to cytokine gene expression (Weinstein S L, et al. J Immunol (1993) 151:3829-33; Stefanova I, et al. Science (1991) 254:1016-7). The requirement of tyrosine (protein) phosphorylation in the activation of tumoricidal properties in murine macrophages by JBT3002 was demonstrated by the use of two specific PTK inhibitors, genistein and tyrphostin (Dong Z, et al. J Exp Med (1993) 177:1071-7; Dong Z, et al. J Leukoc Biol (1993) 53:53-60; Dong Z, et al. J Immunol (1993) 151:2717-25). The inhibition of macrophage activation by MLV-JBT3002 was dose-dependent and could not be reversed by high concentrations of MLV-JBT3002. Interaction of macrophages with MLV-JBT3002 (or LPS) produced phosphorylation of tyrosine on three proteins with masses (39-, 41-, 45-kDa) similar to that of MAP kinases (Dong Z, et al. J Leukoc Biol (1993) 53:53-60). Phosphorylation occurred as early as 20 min after exposure of macrophages to MLV-JBT3002, suggesting that the interaction of liposome-bound JBT3002 with a macrophage surface component may trigger tyrosine phosphorylation. Indeed, preliminary data from our laboratory show that free JBT3002 (not entrapped in liposomes) can activate human monocytes to become cytotoxic and that the binding of JBT3002 to the monocyte surface is independent of binding to CD14, which is, in contrast, mandatory for LPS-induced activity (Weinstein SL, et al. J Immunol (1993) 151:3829-33; Wright S D, et al. Science (1990) 249:1431-2).

Activated macrophages can produce more than 100 distinct molecules ranging in size from 32 kDa (superoxide anion) to 400 kDa (fibronectin) (Nathan C F. J Clin Invest (1987) 78:319-30). The production of so many diverse molecules accounts for the role of macrophages in multiple biological processes that range from mitogenesis and tissue repair to destruction of tumor cells or microorganisms (Fidler I J. et al Encyclopedia of Cancer, vol II. Orlando, Fla.: Academic Press, 1997;1031-41). A major diffusible mediator that can produce death in adjacent tumor cells is NO, which is regulated by the activity of iNOS (Xie K, et al. Cancer Res (1995)55:3123-31; XieK, et al. J Exp Med (1995) 181:1333-44; Dinney C P N, et al. Principles and Practice of Genitourinary Oncolog, Philadelphia: Lippincott-Raven, 1996;17-24).

The mechanism by which MLV-JBT3002-activated macrophages mediated tumor cell lysis is by the production of NO. This conclusion is based on the results of two studies. First, NMA, a specific inhibitor of iNOS, blocked production of NO and tumoricidal properties I macrophages incubated with MLV-JBT3002. Second, subsequent to interaction with JBT3002, macrophages harvested from iNOS knockout mice (MacMiking J D, et al. Cell (1995) 81:641-50) did not produce NO and were not cytotoxic against tumor cells. There were discernible differences between activation of macrophages by LPS and MLV-JBT3002. MLV-JBT3002 equally activated tumoricidal properties in macrophages from both LPS-responsive (C3H/HeN) and LPS-nonresponsive (C3H1H3J) mice, whereas LPS did not. These data agree with results of studies with human monocytes showing that, in contrast to LPS, activation with JBT3002 is independent of serum-binding protein and binding to CD14. Wvhether the in vivo administration of MLV-JBT3002 will not produce adverse side effects associated with LPS or lipid A (Niewoehner D E, et al. J Appl Physiol (1987) 63:1979-86; Arbibe L, et al. J Immunol (1997) 159:391-400) is now under active investigation.

Since NO appeared to be the major cytotoxic molecule that mediated lysis of tumor cells by MLV-JBT3002-activated macrophages, its production as a measure of tumoricidal activation was monitored. These data indicate that tyrosine phosphorylation was an initial step in the MLV-JBT3002-mediated activation cascade, triggered downstream signals that in turn lead to expression of diverse genes and production of many molecules, including NO radicals. The kinetics data suggest that the lag period between exposure of macrophages to MLV-JBT3002 and production of biologically significant levels (>20 μM) of NO and, hence, tumoricidal activity, is 8-12 h.

The biological half-life of MLV-entrapped immunomodulators can determine the schedule of in vivo administration. The finding that once activated by interaction with MLV-JBT3002, macrophages were highly cytotoxic for 2-3 days and could be reactivated by a second exposure to MLV-JBT3002 suggests that in vivo administration need not be given more often than three times weekly.

In summary, herein the new synthetic JBT3002 lipopeptide entrapped in PC/PS liposomes is shown to be a potent activator of tumoricidal properties of murine macrophages by a mechanism that differs from LPS. These data highly support the in vivo use of MLV-JBT3002 to enhance host resistance to infections and cancer.

### EXAMPLE 2

### MATERIALS AND METHODS

Reagents

Eagle's minimum essential medium (EMEM), Hanks' balanced salt solution (HBSS), and fetal bovine serum (FBS) were purchased from Life Technologies (Grand Island, N.Y.). Recombinant mouse IFN-γ (specific activity 1 10⁵ U/mg protein) was obtained from Genentech (San Francisco, Calif.). Phenol-extracted Salmonella lipopolysaccharide (LPS) was purchased from Sigma Chemical, Inc. (St. Louis, Mo.). JBT3002 was obtained from Jenner Technologies (San Raphael, Calif.). Genistein, PD98059, calphostin-C, and H-89 were purchased from Calbiochem-Novabiochem Int. (San Diego, Calif.). The enzyme-linked immunosorbent assay (ELISA) kits for mouse TNF-α, IL-α,, IL-6, IL-10, and GM-CSF were purchased from R&D Systems, Inc. (Minneapolis, Minn.). 1′,2′-Dioleoyl-sn-glycero-3-phospho-L-serine monosodium salt (PtdSer) and 1-palmitoyl-2-oleoyl-sn-glycero-3-phosphocholine (PtdCho) were synthesized at Ciba-Geigy, Ltd. (Basel, Switzerland). (van Hoogevest P, et al. Liposomes in the therapy of infectious diseases and cancer. Liss, New York, 1989;453-466) All reagents, except for LPS, were free of endotoxin as determined by the Limulus aboebocyte assay (detection limit 0.125 ng/ml) acquired from Associates of Cape Cod, Inc. (Walpole, Mass.).

Preparation of Liposomes

PC (175 mg), PS (75 mg), and JBT3002 0.1 mg) were dissolved in chloroform. The lipids, with or without immunomodulators, were dried as a thin layer onto the glass by rotating the tube under a gentle stream of nitrogen gas. Residual chloroform was removed by incubating the tubes in a vacuum overnight at room temperature. Multilamellar liposomes were prepared by hydrating the lipid film with HBSS, followed by vigorous shaking for 5 minutes using a vortex shaker. (Schroit AJ, et al. Cancer Res (1982) 42:161-167) The liposomes were diluted into EMEM for addition to macrophage cultures.

Animals

Specific, pathogen-free, female C57BL/6 mice were purchased from the Jackson Laboratory (Bar Harbor, Me.). The mice were used when they were 8-12 weeks old. Animals were maintained according to institutional guidelines in facilities approved by the American Association for Accreditation of Laboratory Animal Care and in accordance with current United States Department of Agriculture, Department of Health and Human Services, and the National Institutes of Health regulations and standards.

Isolation and Activation of Macrophages

Peritoneal exudate macrophages (PEM) were collected by peritoneal lavage from mice given an intraperitoneal injection of 1.5 ml of thioglycollate broth (Baltimore Biological Laboratory, Cockeysville, Md.) 4 days before harvest.(Saiki I, et al. J Immunol (1985) 135:684 688; Dong Z, et a.l J Leukoc Biol (1993) 53:53-60; Dong Z, J et al. J Exp Med (1993) 177:1071-1077) The PEM were washed in CA²⁺- and MG²⁺-free HBSS and resuspended in serum-free medium: 1×10⁵ cells were plated into each 38-mm² well of 96-well culture plates (Falcon Plastics, Oxnard, Calif.). After a 90-minute incubation, the nonadherent cells were removed by washing them with medium. More than 98% of the adherent cell populations were macrophages according to morphology and phagocytic criteria. (Saiki I, et al. J Immunol (1985) 135:684 688)

ELISAs for Cytokines

PEM in 96-well plates at a density of 10⁵ cells/well/200 μl of medium were treated as indicated in Results. The culture supernatants were harvested and used immediately for the cytokine assays or stored at −70° C. For cell lysates, supernatants were removed and macrophages were frozen-thawed in fresh medium. Specific cytokines in the supernatants or cell lysates (diluted at 1:5 or 1:10) were measured by ELISA kits according to manufacturer's instructions.

Assay for Nitrite Production

The nitrite concentration in culture supernatants was determined by a microplate assay as described previously. Briefly, 50-μl samples harvested from PEM-conditioned medium were treated with an equal volume of Griess reagent (1% sulfanilamide, 0.1% naphthylene diamine dihydrochloride, and 2.5% H₃PO₄) at room temperature for 10 minutes. The absorbance at 540 nm was monitored with a microplate reader. The nitrite concentration was determined using sodium nitrite as a standard.

Northern Blot Analysis

PEM were plated into 150-nm dishes at 5×10⁷ cels/plate. mRNA was extracted using the FastTrack mRNA isolation kit (Invitrogen, San Diego, Calif.) from PEM cultured in medium alone or with different agents as indicated in the results. mRNA was electrophoresed on 1% denaturing formaldehyde-agarose gel, electrotransferred to GeneScreen nylon membrane (DuPont Co., Boston, Mass.), and UV cross-linked with 120,000 μJ/cm² using a UV Stratalinker 1800 (Stratagene, LA Jolla, Calif.). Cytokines and GAPDH mRNA were detected using cDNA probes of mouse TNF-α, IL-1α, IL-6, GM-CSF, and rat glyceraldehyde-3-phosphate dehydrogenase (GAPDH) labeled by nick translation with [α-³²P]CTP. Hybridizations were performed as described previously. (Kumar R, et al J Immunol (1996) 157:5104-5111). Nylon filters were washed at 55-60° C. with 30 mM NaCl, 3 mM sodium citrate (pH 7.2), and 0.1% SDS.

Densitometric Quantitation

Expression of cytokine genes was quantified by densitometry of autoradiograms using an Image Quant software program (Molecular Dynamics, Sunnyvale, Calif.). The value for each sample was calculated as the ratio of the average areas of cytokine-specific mRNA transcripts to the GAPDH mRNA transcript in the linear range of the film.

Statistical Analysis

All experimental results were analyzed for statistical significance by the use of the two-tailed Student's t-test.

RESULTS

Expression of Cytokines in Macrophages

In Example 1, liposome-encapsulated JBT3002 was shown to induce production of nitric oxide (NO) in murine macrophages and hence activate the cells to lyse tumorigenic target cells. Since the tumoricidal activity of monocytes/macrophages is mediated by secretory products, including cytokines, whether JBT3002 encapsulated in liposomes induced the production of TNF-α, IL-1α, IL-4, IL-6, IL-10, and GM-CSF by murine macrophages was evaluated. PEM were incubated for 24 hours in medium containing different concentrations of liposomes--JBT3002 (0.1 mg/300 μmol phospholipid) in the presence or absence of 20 U/ml IFN-γ.

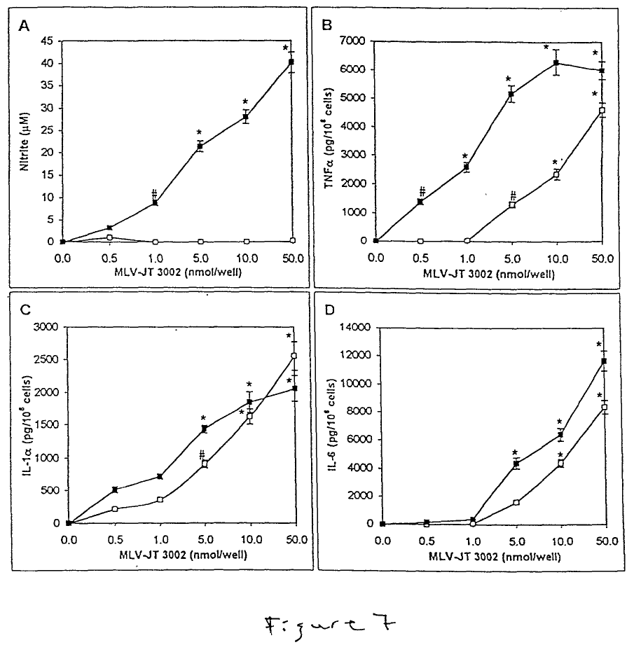

Treatment of PEM with MLV-JBT3002 in the presence of 10 U/ml IFN-γ induced the production of NO in a dose-dependent manner (FIG. 7A). Production of TNF-α (FIG. 7B), IL-1α (FIG. 7C), and IL-6 (FIG. 7D) by liposome JBT3002-activated macrophages did not require the presence of IFN-γ, although at the lower concentrations of JBT3002, IFN-γ did enhance production of TNF-α. The culture supernatants did not contain significant levels of IL-4, IL-10, or GM-CSF (data not shown).

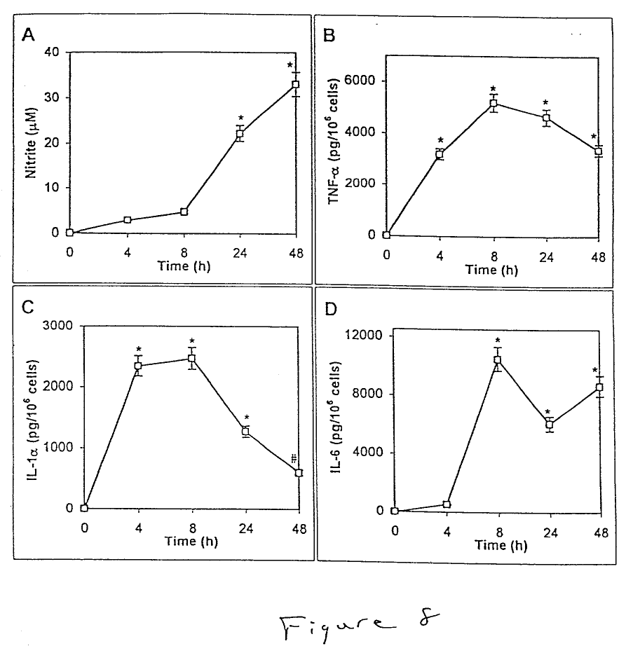

In the next set of experiments, the time course of cytokine production by PEM was monitored. Significant levels of TNF-α and IL-1α were detected by 4 hours after incubation of PEM with 50 nmol/well MLV-JBT3002 (0.1 mg/300 μmol lipid). The production-release of TNF-α and IL-1α reached a plateau by 8 hours (FIGS. 8B and 8C). The production-release of IL-6 also peaked at 8 hours after exposure of PEM to MLV-JBT3002 (FIG. 8D). IFN-γ did not alter the kinetics of cytokine production-release (data not shown).

Induction of Cytokine mRNA

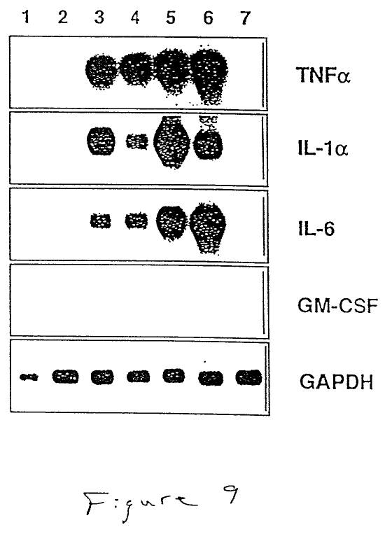

PEM were treated for 4 hours with LPS (100 ng/ml) or MLV-JBT3002 (0.1 mg/300 μmol lipids) in the presence or absence of IFN-γ (10 U/mi). mRNA was extracted and analyzed for cytokine expression by northern blotting. Control PEM incubated with medium alone, medium containing IFN-γ, or medium with MLV containing HBSS (FIG. 9, lanes 1, 2, and 7) did not express any detectable levels of mRNA for TNF-α, IL-1α, IL-6, and GM-CSF. LPS (lanes 3 and 4) and MLV-JBT3002 (lanes 5 and 6) induced the expression of mRNA for TNF-α, IL-1α, and IL-6 in the PEM. The presence of IFN-γ (lanes 4 and 6) did not increase the expression of mRNA for these cytokines in comparison with PEM treated with LPS or MLV-JBT3002 in the absence of IFN-γ (lanes 3 and 5). MLV-JBT3002 also induced the expression of GM-CSF, albeit to a low level (lane 5). This expression did not correlate with production of detectable levels of protein (data not shown).

Induction of Cytokines by JBT3002 is Serum-independent

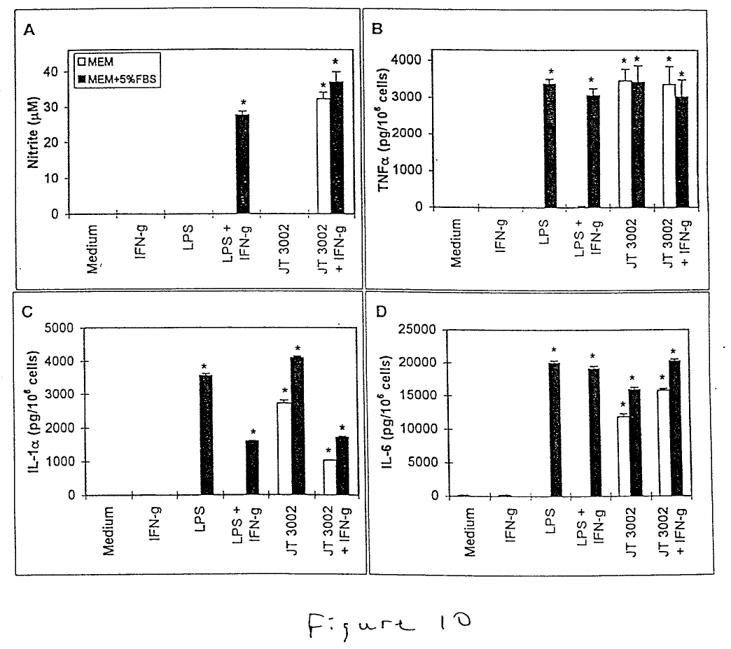

Since the activation of monocytes/macrophages by LPS requires a serum LPS-binding protein, (Wright S D, et al. Science (1990) 249:1431-1439; Schumann R R, et al. Science (1990) 249:1429-1431) whether the activation of PEM by JBT3002 was also serum-dependent was evaluated. PEM were incubated in serum-free or serum (5% FBS)-supplemented EMEM containing IFN-γ, LPS, LPS plus IFN-γ, MLV-JBT3002, or MLV-JBT3002 plus IFN-γ. LPS plus IFN-γ generated production of NO (FIG. 10A), TNF-α (FIG. 10B), IL-1α (FIG. 10C), and IL-6 (FIG. 10D) only in the presence of serum. Activation of PEM by MLV-JBT3002 alone or in the presence of IFN-γ (to produce NO, TNF-α, IL-1αm abd IL-6) was independent of serum.

Involvement of Protein Tyrosine Kinase in the Activation of PEM by JBT3002

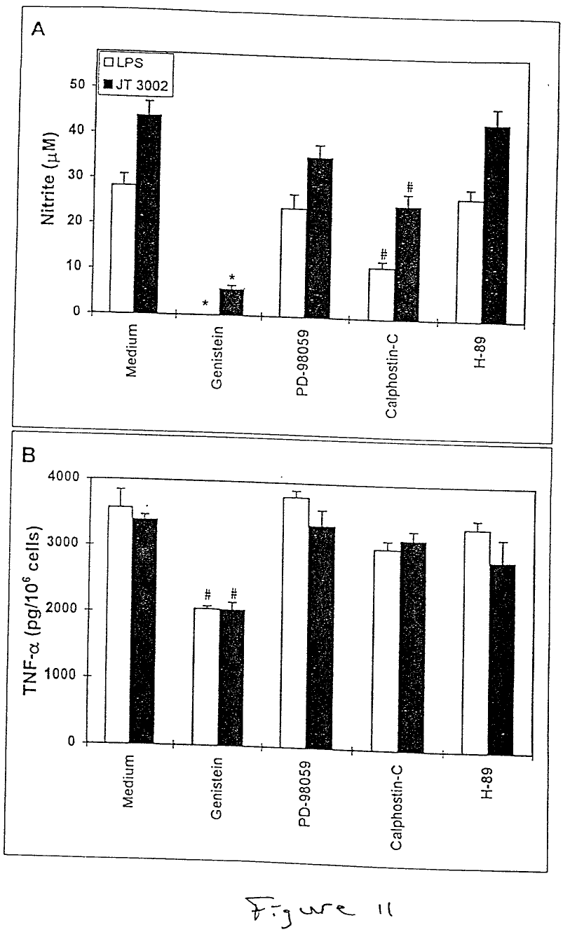

The activation of macrophages by LPS requires tyrosine phosphorylation of different proteins, (Dong Z, et a.l J Leukoc Biol (1993) 53:53-60; Dong Z, J et al. J Exp Med (1993) 177:1071-1077; Ding A E, et al. J Immunol (1993) 151:5596-5602; Weinstein S L, et al. J Biol Chem (1992) 267:14955-14962) activation of MAP kinases, (Dong Z, J et al. J Exp Med (1993) 177:1071-1077; Arditi M, et al. J Immunol (1995) 155:3993-4003; Liu M K et al J Immunol (1994) 153:2642-2652) and protein kinase C (PKC). (Paul A, et al. Br. J Pharmacol (1995) 114:482488; Shinji H, et al. J Immunol (1994) 153:5760-5771; Novotney M, et al. Biochemistry (1991) 30:5597-5604). To determine whether activation by JBT3002 is mediated by these kinases, PEM with EMEM containing the MAP kinase kinase (MEK) inhibitor PD-98059, (Dudley D T, et al. Proc Natl Acad Sci USA (1995) 92:7686-7689) the tyrosine kinase inhibitor, genistein, (Constantinou A, et al. Proc Soc Exp Biol Med (1995) 208:109-115) the protein kinase C inhibitor, calphostin-C, (Jarvis W D, et al. Cancer Res (1994) 54:1707-1714) and the protein kinase A inhibitor H-89 (Findik D, et al. J Cell Biochem (1995) 57:12-21) were each incubated for 20 minutes prior to the addition of LPS or MLV-JBT3002. After 24 hours, the PEM culture supernatans were assayed for nitrite content (FIG. 11A) or TNF-α (FIG. 11B). At the concentrations used, none of the compounds were toxic to macrophages (data not shown). PD-98059 did not alter the production of TNF-α or NO induced by either LPS or JBT3002. Genistein significantly inhibited the production of TNF-α and NO by PEM treated with LPS or JBT3002. Neither calphostin-C nor H-89 had a significant effect on the production of TNF-α, although calphostin-C did inhibit NO production in PEM treated with LPS or MLV-JBT3002. The inhibition of cytokine production by Genistein occurred at the level of mRNA as assessed by northern blot analysis (data not shown).

DISCUSSION

The present results demonstrate that JBT3002, a new synthetic lipopeptide of the outer wall of a gram-negative bacterium, is a potent activator of inflammatory cytokines in murine macrophages. Activated macrophages can produce more than 200 distinct molecules ranging in size from 32 dalton (superoxide anion) to 400 kDa (fibronectin). (Nathan C F. J Clin Invest (1987) 78:319330). The diversity of these molecules accounts for the multifaceted role of macrophages, ranging from mitogenesis and tissue repair to destruction of tumor cells and microorganisms. (Fidler I J. Adv Pharmacol (1994) 30:271-326; Fidler I J. Cancer Res (1985) 45:4714-26). The potentiation of cytokine production by macrophages using synthetic immunomodulators such as JBT3002 may therefore improve the clinical management of cancer and infectious diseases. For these studies, JBT3002 was encapsulated in multilamellar liposomes composed of PC and PS 7:30 molar ratio). Consistent with previous reports, (Asano T, et al. J Immunother (1993) 14:268-292; Schroit A J, et al. Cancer Res (1982) 42:161-167) herein is shown that these liposomes allow for efficient activation of macrophages to produce NO and cytokines.

Macrophage activation by LPS requires LPS-binding protein found in serum (Wright S D, et al. Science (1990) 249:1431-1439; Schumann R R, et al. Science (1990) 249:1429-1431) which these studies confirmed. In contrast, however, the activation of macrophages by the lipopeptide JBT3002 did not require serum proteins. Activation of protein kinases, especially protein tyrosine kinases and PKC, are important for intracellular signaling of various macrophage-activating agents. (Dong Z, et a.l J Leukoc Biol (1993) 53:53-60; Dong Z, Jet al. JExp Med(1993) 177:1071-1077; Ding AE, et al. J Immunol (1993) 151:5596-5602). To determine the role of various kinases in JBT3002-mediated macrophage activation, different inhibitors of protein kinases were used and found that, as is the case for LPS, JBT3002-induced signaling involves protein tyrosine kinases. PKC and PKA are not involved in the cytokine induction by either LPS or JBT3002, but the PKC inhibitor calphostin-C inhibited NO production in the presence of IFN-γ. These results confirm the role of PKC in IFN-γ-mediated signal transduction. (Celada A, et al. J Immunol (1986) 137:2373-2379).

In conclusion, the synthetic lipopeptide JBT3002 induced TNF-α, IL-1α, and IL-6 production in mouse peritoneal macrophages by a mechanism that is similar to though distinct from LPS. These studies further support the systemic administration of JBT3002 to enhance host resistance to infections and cancer.

### EXAMPLE 3

A primary function of monocytes/macrophages is to discriminate between “self” and “altered self” and thus participate in host defense against microorganism and cancer. This function requires monocyte/macrophage activation, which is achieved subsequent to interaction with lymphokines such as IFN-γ and whole microorganism or their products such as LPS, cell wall skeleton, and bacterial components such as muramyl dipeptide. The activation of bactericidal-tumoricidal properties in macrophages by lymphokines and bacterial components frequently occurs in sequence: for example, IFN-γ primes macrophages to respond to a second signal such as LPS. Activated monocytes/macrophages produce more than 100 distinct molecules, including TNF-α, IL-1β, IL-6, and prostaglandins, and different stimuli can induce the release of different products.

While the antitumor activity of LPS and lipid A, the active component of LPS, was established in a variety of tumor models, their therapeutic application, unfortunately, went unrealized, partly because of dose-limiting side effects. For this reason, many attempts have been made to develop synthetic activators of monocytes/macrophages, which led to the discovery of a series of compounds that can render monocytes/macrophages tumoricidal. These compounds include muramyl dipeptide, muramyl tripeptide phosphatidylethanolamine (MTP-PE), and the lipopeptide, CGP31362.

Efficient in situ activation of macrophages can be achieved by the encapsulation of immunomodulators within phospholipid liposomes. The systemic administration of liposomes with MTP-PE has produced regression of metastases in murine tumor systems, dogs with spontaneous osteogenic sarcoma, and increased disease-free survival in children with chemotherapy-resistant osteogenic sarcoma lung metastases. Whether different synthetic molecules would produce a more effective therapy remained unclear.

The incubation of human monocytes with MTP-PE or lipopeptide CGP31362 induced production of different cytokines. Moreover, liposomes containing CGP31362 produced superior tumoricidal activation of macrophages leading to regression of metastases in murine systems. The usefulness of the lipopeptide CGP31362, however, has been limited by its solubility properties, prompting the design of analogues.

### MATERIALS AND METHODS

Reagents

Eagle's MEM (EMEM), HBSS, and FBS were purchases from M. A. Bioproducts (Walkersville, Md.). Human recombinant interferon-gamma (IFN-γ) (sp. act., 5.2×10⁷ U/mg protein) was the generous gift of Genentech, Inc. (South San Francisco, Calif.), and the phenol-extracted Salmonellia LPS was purchased from Sigma Chemical, Inc. (St. Louis, Mo.). The ELISA kits for human TNF-α, IL-1β, and IL-6 were purchased from BioSource International (Camarillo, Calif.). [³ H]TdR (sp. act., 2 Ci/mmol) was purchased from ICN Biomedicals (Costa Mesa, Calif.). JBT3002 was generously provided by Jenner Technology (San Ramon, Calif.). Human CD14-specific hybridoma 3C10 was obtained from the American Type Culture Collection (Rockville, Md.). Neat ascites fluid produced in BALB/c mice was used. Monoclonal antiphosphotyrosine antibody 4G10 was purchased from UBI (Lake Placid, N.Y.). JNK-specific monoclonal antibody 333.1 was raised against JNK1 and ascitic fluids used in Western blot analysis. Rabbit anti-activated MAP kinase antibody was purchased from Promega (Madison Wis.). All reagents used in tissue culture, except LPS, were free of endotoxin as determined by the Limulus amebocyte lysate assay (sensitivity limit of 0.125 ng/ml) (Associates of Cape Code, Walpole, Mass.).

Preparation of JBT3002

Free-form JBT3002: JBT3002 was suspended in HBSS at 1 mg/ml, sonicated for 5 min, and stored at 4° C. It was vortexed prior to each experiment. Liposomeencapsulated JBT3002: PC (175 mg), PS (75 mg), and JBT3002 (0.125, 0.25, 0.5, or 1.0 mg) were dissolved in chloroform. The clear solution was sterile-filtered through a Gelman-TF-200 (0.2-μm filter). Aliquots of 1 ml containing phospholipids with or without immunomodulators were retroevaporated under a stream of nitrogen gas. The tubes with dry film were in cubated overnight in a vacuum chamber at room temperature. Multilamellar liposomes were prepared by hydration of the lipid film with HBSS and high-speed agitation on a vortex for 6 min as in Example 2. The liposomes were diluted into Eagle's MEM before addition to monocyte cultures.

Tumor Cell Lines

A375SM human melanoma cells (Ichinose, Y. et al. Cancer Immunol. Immunother. (1988) 27:7) were maintained as monolayer cultures in EMEM supplemented with vitamins, sodium pyruvate, nonessential amino acids, L. glutamine, and 10% FBS. The cell line was free of Mycoplasma and pathogenic mouse viruses.

Isolation of Human Monocytes

Blood-cells buff coats were obtained on the day of collection from the Gulf Coast Regional Blood Center (Houston, Tex.). The buffy coats were diluted with HBSS and layered on to 15 ml of prescreened entotoxin-free lymphocyte separation medium (Ficoll-Hypaque; density: 1.077). After 10 min of centrigugation at 1500×g, the mononuclear fractions were collected, washed once, and resuspended in 20 ml of elutriation medium (2% human albumin-100 U/mi penicillin and 100 μg/ml streptomycin in PBS). Monocyte-rich fractions were isolated by countercurrent elutriation using a JE-6B elutriation rotator (B3eckman) as described in detail previously (Fidler, I. et al. Prog. Clin. Biol. Res. (1989) 288:169). At a speed of 3000 rpm and flow rate of 41 ml/min, the monocyte fraction was obtained; it contained >90-95% monocytes as identified by nonspecific esterase staining morphological examination; they were >95% viable as measured by the trypan blue exclusion test. The cells were incubated in serum-free EMEM for 18 h prior to assays.

Monocyte-mediated Cytotoxicity

Monocytes plated at a density of 1×10⁵ cells/38-mm² well of 96-well plates were incubated at 37° C. for 18-24 h with medium or with medium containing different concentrations of free-form or MLV-JBT3002 or LPS in the presence or absence of 10 U/ml human IFN-γ. Monocyte-mediated cytotoxicity was assessed by measuring the release of radioactivity from DNA of prelabeled target cells as described previously (Dong, Z. et al. J Immunol. (1993) 151:2717). Briefly, A375SM cells in the exponential phase of growth were incubated for 24 h in supplemented EMEM containing 0.2 μCi/ml [³ H]TdR (sp. act., 2 Ci/mmol). The tumor cells were harvested by a brief trypsinization (0.25% trypsin and 0.02% EDTA), washed, resuspended in supplemented EMEM, and plated into wells containing monocytes (10×10⁴ tumor cells/well). After a 72-h coincubation, the cultures were washed twice with PBS, and adherent viable cells were lysed with ) 0.1 ml of 0.1 N NaOH. The lysates were harvested with a Harvester 96 (Tomtec, Orange, Conn.) and counted in a liquid scintillation counter. The cytotoxic activity of monocytes was calculated as follows:

Cytotoxicity (%)=(A−B)/A×100

where A=cpm in cultures of control monocytes and target cells, and B=cpm in cultures of treated monocytes and target cells.

ELISAs for TNF-α, lL-1β and IL-6

After overnight incubation in serum-free EMEM, monocytes plated at the density of 1×10⁵/38-mm²well /200 μl of EMEM (96-well plates) were treated as indicated in the Results section. The culture supernatants were harvested and used immediately or stored at −70° C. The supernatants were diluted at 1:5 or 1:10 and assayed for cytokines using ELISA kits according to the manufacturer's instructions.

Western Blot Analysis

Monocytes (2.5×10⁶/30-mm diameter dish) incubated at 37° C. were treated with different concentrations of LPS or LPS or JBT3002 as indicated in the Results section. After two washes with PBS containing 1 mM Na₃ VO₄ and 5 mM EDTA, the cells were scraped into 0.1 ml lysis buffer (1% Triton X-100, 20 mM Tris pH 8.0, 137 MM NaCl, 10% glycerol, 1 mM Na₃ VO₄, 2 mM EDTA, 1 mM PMSF, 20 μM leupeptin, 0.15 U/ml aprotinin). The lysate was placed on ice for 20 min and then centrifuged at 14,000 rpm for 10 min at 4° C. The samples (50 μg) were mixed with sample buffer (62.5 mM Tris/HCl, pH 6.8, 2.3% SDS, 100 mM DTT, and 0.05% bromophenol blue), boiled and, separated on 10% SDS-PAGE. The protein was then transferred onto 0.45 μm nitrocellulose membranes. The filter was blocked with 3% BSA and 1% ovalbumin (ICN Biomedicals, Inc.) in TBS (20 mM Tris/HCl, pH 7.5, 150 mM NaCl), probed with antibodies as indicated in the Results in TTBS (TBS containing 0.1% Tween 20), incubated with a second antibody in the buffer, and visualized by the ECL Western blotting detection system (Dong, Z., et al. J Exp. Med. (1993) 177:1071; Dong, Z. et al. J. Leukoc. Biol. (1993) 58:725).

RNA Isolation and Northern Blot Analyses

Monocytes were plated at a density of 1.2×10⁷ cells/100-mm dish. Total RNA was extracted using Tri reagent™ kit according to the manufacturer's instructions (Molecular Research Center, Inc., Cincinnati, Ohio). For northern lot analyses, 10-20 μg of total RNA was separated in 1% denaturing formaldehyde-agarose gels, transferred to GeneScreen nylon membrane, and UV cross-linked with 120,000 μJ/CM²using a UV Stratalinker 1800. Cytokine and GAPDH and mRNA were detected using cDNA probes of Human TNF-α, IL-1β, IL-6 and rat glyceraldehyde-3-phosphate dehydrogenase (GAPDH) labeled by nick translation with α-³²P-CTP. Hybridizations were performed as described (Dong, Z. et al. J Natl. Cancer Inst. (1994) 86:913). Filters were washed at 55-60° C. with 30 mM NaCl, 3 mM sodium citrate (pH 7.2), 0.1% SDS.

Statistical Analysis

The experimental results were analyzed for their statistical significance by the two-tailed Student's t test.

Results

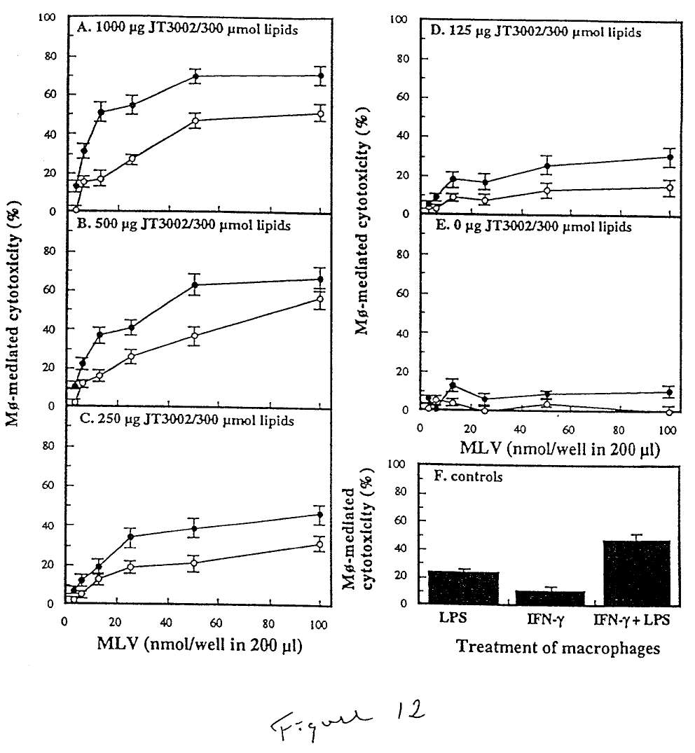

Activation of tumoricidal properties in blood monocytes by MLV-JBT3002 In the first set of experiments, whether liposome-encapsulated JBT3002 could activate monocyte-mediated tumoricidal activity were determined. Human peripheral blood monocytes were incubated for 18-24 h with various concentrations of MLV containing JBT3002 at different JBT3002/phospholipid ratios in the presence or absence of 10 U/ml human IFN-γ. The treated monocytes were washed and p³H]TdR-labeled A375SM melanoma cells were plated on top of the adherent monocytes. The lysis of the A375SM cells was determined 72 h later. Consistent with previous reports (Nii, A. et al. Lymphokine Res. (1990) 9:113; Jonjic, N. et al. Eur. J Immunol. (1992) 22:2255), nonactivated human monocytes and monocytes incubated with control MLV containing HBSS did not lyse the tumor cells (FIG. 12E), however MLV-JBT3002 did lyse tumor cells in a dose-dependent manner (FIG. 12A-12E). For example, at 50 nmol/well, monocytes treated by MLV containing 1000, 500, 250, and 125 μg JBT3002/300 ,μmol phospholipid lysed 45% (P<0.001), 31% (P<0.01), 19% (P<0.05), and 12% of the tumor cells, respectively. Treatment with 20 U/ml of human IFN-γ alone did not result in monocyte-mediated tumoricidal activity, but it did significantly augment tumoricidal activation of blood monocytes by MLV-JBT3002 (FIGS. 12A-12E) (P<0.01). As positive controls, monocytes were treated with LPS (100 ng/ml) and/or IFN-γ (10 U/ml). As shown in FIG. 12F, 25% and 48% cytotoxicity were observed in monocytes treated with LPS alone (P<0.01) and LPS plus IFN-γ (P<0.001), respectively. These data show that MLV-JBT3002 is a potent activator of tumoricidal properties in human blood monocytes.

Induction of Cytokine Production by MLV-JBT3002

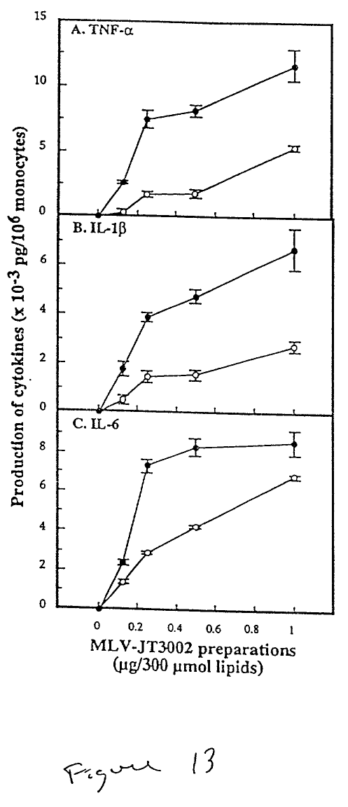

Since the tumoricidal activity of monocytes is mediated by secretory products, including cytokines (Nathan, C. F. J Clin. Invest. (1987) 79:319), the effect of MLV-JBT3002 on the production of three prominent inflammatory cytokines of activated monocytes: TNF-α, IL-1β, and IL-6 was investigated. Monocytes (1×10⁵38-mm² well) were incubated for 24 h with MLV (100 mmol/well) containing various concentrations of JBT3002 in the absence or presence of IFN-γ (10 U/ml). The cytokines in the culture supernatant were measured by ELISA (FIG. 13). MLV-JBT3002 induced the production of TNF-α (panel A), IL-1β (panel B), and IL-6 (panel C) in a dose-dependent manner, and in parallel with tumoricidal activation. IFN-γ alone did not stimulate cytokine production (data not shown), but significantly increased (P<0.01) the production of the three cytokines induced by MLV-JBT3002 (FIG. 13).

Monocyte Activation by Free-form JBT3002

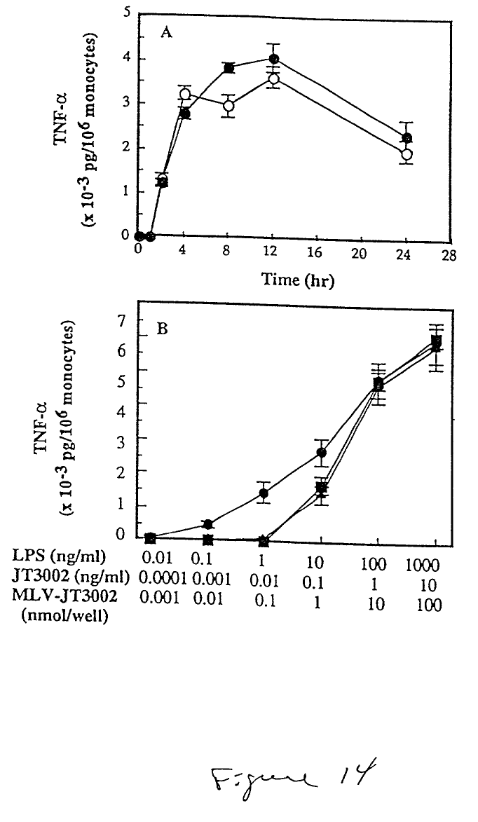

In the next set of experiments, the kinetics of TNF-α production induced by free-form JBT3002 and MLV-JBT3002 were compared. Monocytes were treated for various periods of time with free-form JBT3002 (1 ng/ml) or MLV-JBT3002 (100 nmol/well of 1 mg/300 μmol lipid). TNF-α protein was detected in the culture supernatant of monocytes after 2 h incubation with either free-form JBT3002 or MLV-JBT3002; the levels plateaued at 4-8 h and decreased thereafter (FIG. 14A). There was no significant difference in the kinetics of TNF-α production between monocytes stimulated by free-form JBT3002 and MLV-JBT3002 (FIG. 14A). Next the dose-dependent induction of TNF-α production in monocytes treated for 8 h with LPS, free-form JBT3002, and MLV-JBT3002 was analyzed. LPS induced TNF-α production in a dose-dependent manner in the range of 1-1000 ng/ml (FIG. 14B), but JBT3002 was more potent and activated monocytes in a wider range of concentrations (0.001-10 ng/ml). MLV-JBT3002 activated TNF-α production in a range of 0.1-100 nmol/well (equivalent to 1.5-1,500 ng/ml of JBT3002).

Activation of Human Blood Monocytes by JBT3002 is Serum- and CD14-independent

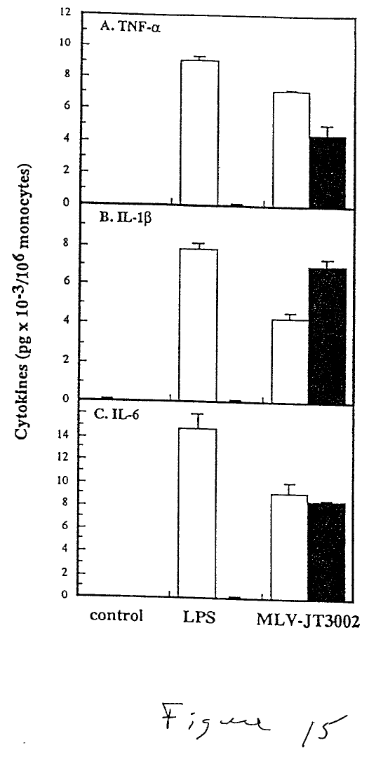

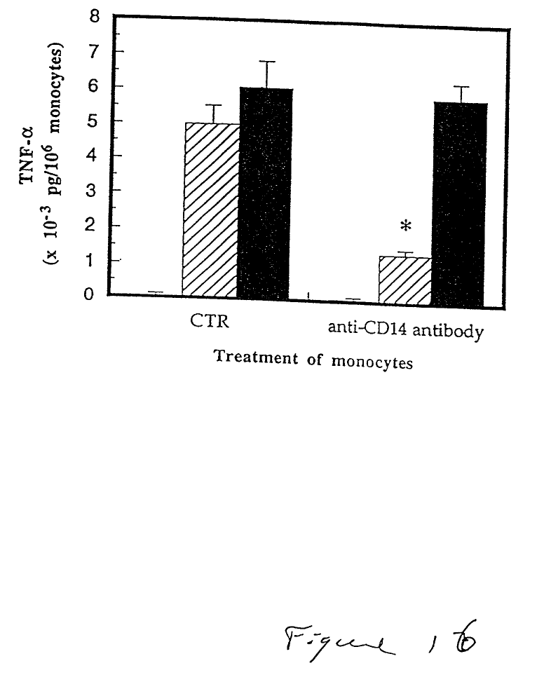

Since activation of monocytes by LPS requires a serum LPS-binding protein (LBP) an is initiated following interaction of LPS-LBP complex with its receptor CD14 on monocytes (Wright, S. D. et al. Science 249:1431), whether serum was required for activation of monocytes by JBT3002 was determined. Monocytes were incubated with LPS or free-form JBT3002 in EMEM containing 5% FBS or serum-free EMEM. In EMEM with 5% FBS, similar amounts of TNF, IL-1, and IL-6 were generated in monocytes activated by LPS (100 ng/ml) and JBT3002 (1 ng/ml) (FIGS. 15A-15C). LPs-induced activation of monocytes was diminished in serum-free EMEM and was reduced by 72% in the presence of 3C10 monoclonal antibody, which is specific to and neutralizes CD14 (FIG. 16). In contrast, production of the cytokines induced by free-form JBT3002 was not significantly altered in the absence of serum (FIGS. 15A-15C) and was not inhibited by the anti-CD14 antibody (FIG. 16).

Expression of Cytokine mRNA

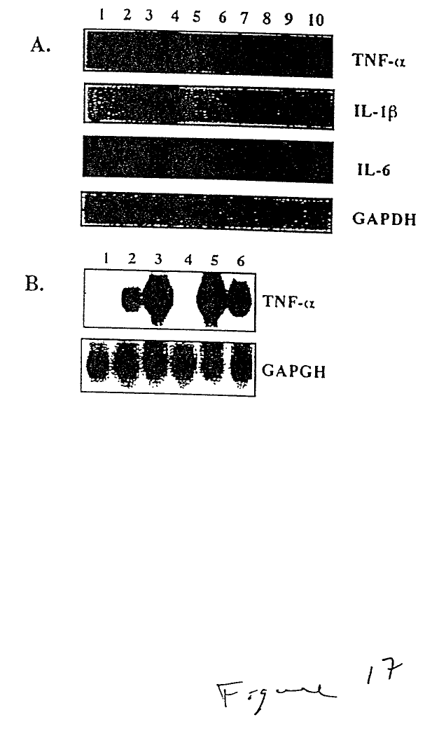

Monocytes were incubated for 1-3 h with LPS (100 ng/ml), MLV-JBT3002 (500 nmol/ml of 1 mg JBT3002/300 μmol lipids), and JBT3002 (I ng/ml) in the presence or absence of 10 U/ml IFN-γ. Total cellular RNA was then extracted and analyzed by northern blotting. As shown in FIG. 17, resting monocytes and monocytes treated with MLV-HBSS constitutively expressed low levels of TNF-α mRNA and did not express detectable levels of steady-state mRNAs for IL-1 and IL-6 (FIG. 17A, lanes 1 and 5). Expression of TNF-α, but not IL-1β and IL-6, was increased by the presence of IFN-γ (FIG. 17A, lanes 2 and 6). High levels of mRNA for TNF-α, IL-1β, and IL-6 were expressed in cells treated with LPS (FIG. 17A, lane 3), MLV-JBT3002 (FIG. 17A, lane 7), or free-form JBT3002 (FIG. 17, lane 9). Expression of TNF-α, but not IL- I and IL-6, induced by LPS, MLV-JBT3002, or free-form JBT3002 was augmented by the presence of IFN-γ (FIG. 17A, lanes 4, 8, and 10). In agreement with the production of TNF-α protein, a significant reduction of steady-state TNF-α mRNA was noted in monocytes stimulated by LPS in serum-free medium (FIG. 17B, lane 2) as compared with that in serum containing medium (FIG. 17B, lane 5). Induction of TNF-α mRNA by JBT3002 did not require serum, as evidenced by high levels of TNF-α mRNA whether in the absence (FIG. 17B, lane 3) or presence (FIG. 17B, lane 6) of serum.

Activation of Intracellular Signaling Pathway

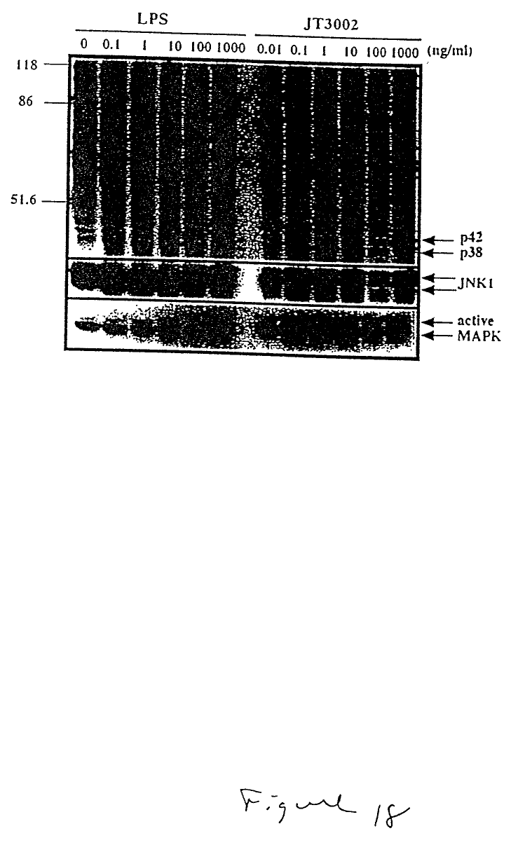

Treatment of macrophages and monocytes with LPS triggers many intracellular signaling pathways. Among them are protein tyrosine phosphorylation (Wright, S. D. et al. Science (1990) 249:1431; Weinstein, S. L. et al. J Immunol. (1993) 151:3829; Stefanova, I. et al. Science (1991) 254:1016), and activation of JNK1 (Hambleton, J. et al. Proc. Natl. Acad. Sci. USA (1996) 93:2774) and MAP kinases (Dong, Z., et al. J. Exp. Med. (1993) 177:1071; Liu, M. K. et al. J. Immunol. (1994) 153:2642; Arditi, M. et al. J. Immunol. (1994) 155:3994), which may be involved in production of cytokines and tumoricidal activation of monocytes-macrophages (Dong, Z., et al. J. Exp. Med. (1993) 177:1071; Dong, Z. et al. J Immunol. (1993) 151:2717; Dong, Z. et al. J. Leukoc. Biol. (1993) 53:53). Whether JBT3002 could activate these signaling pathways was investigated. After incubation of monocytes for 20 min with increasing concentrations of LPS or free-form JBT3002, lysates were prepared and analyzed by Western blotting. As shown in FIG. 18, treatment of monocytes with LPS induced tyrosine phosphorylation of proteins with apparent molecule masses of 42 and 38 kDa, a JNK1 band shift, and activation of MAP kinase (detected using an antibody specific to activated Erks) in a dose-dependent manner. Significant tyrosine phosphorylation and MAP kinase activation, and JNK1 band shift were observed in cells treated with 10 ng/ml of LPS; the JNK1 band shift occurred at 100 ng/ml. Similar patterns of tyrosine phosphorylation, JNK1 band shift, and MAP kinase activation were observed in monocytes incubated with JBT3002 (FIG. 18). Consistent with the induction of cytokine production, JBT3002 was significantly more potent than LPS in triggering these intracellular signaling pathways (FIG. 18). JNK1 kinase activity assessed using GST-c-Jun as substrate showed that JNK1 band shift induced by LPS and JBT3002 correlated with activation of the kinase (data not shown).

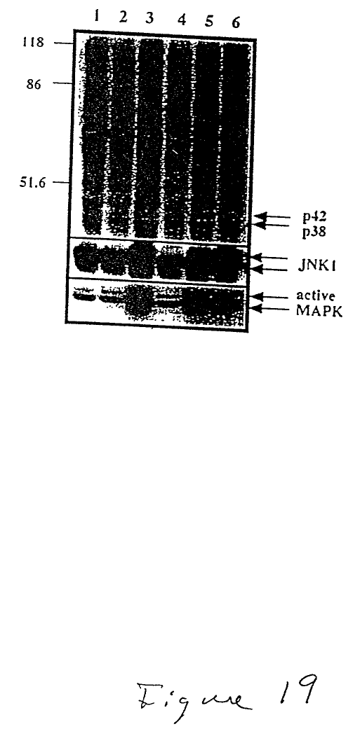

Induction of tyrosine phosphorylation, JNK1 band shift, and MAP kinase activation by LPS required the presence of serum (FIG. 19, lane 2 [serum-free] vs lane 5 [5% FBS]), whereas the same responses in monocytes stimulated by JBT3002 did not (FIG. 19, lane 3 vs 6).

Discussion

The purpose of this example was to investigate whether JBT3002, a new synthetic analogue of a lipoprotein from the outer wall of gram-negative bacteria could activate production of inflammatory cytokines and tumoricidal properties of human blood monocytes. Previous studies indicated that MLV composed of PC:PS (molar ratio 7:3) are preferentially recognized by monocytes/macrophages and that immune modulators encapsulated in these phospholipid liposomes are significantly more potent in the in vivo activation of monocytes/macrophages than immunomodulators administered alone (Fidler, I. J. Adv. Drug Del. Rev. (1988) 2:69). These present results demonstrate that tumoricidal properties and expression of the inflammatory cytokines TNF-α, IL-1β, and IL-6 were activated in monocytes by MLV-JBT3002 in a dose-dependent manner. This activation was augmented by the presence of recombinant human IFN-γ. Moreover, JBT3002 was more potent than LPS in the activation of monocytes.

To determine whether phagocytosis of MLV-JBT3002 is necessary for its action, dose-dependent response and kinetics of TNF-α induced by MLV-JBT3002 and free-form JBT3002 were studied. These data show that free-form JBT3002 was even more potent than MLV-JBT3002 in the induction of cytokine gene expression. In addition, activation of monocytes by MLV-JBT3002 and free-form JBT3002 followed the same kinetics. TNF-α protein was found in culture supernatants after a 2-h stimulation and reached a plateau 4-8 h later. Since maximal internalization of liposome requires 8-16 h, these data suggest that activation of monocytes may not require phagocytosis of MLV-JBT3002 and might be induced by interaction of the monocytes with micellar JBT3002. The use of JBT3002 in vivo may be greatly enhanced by its encapsulation in phospholipid liposomes.

Previous studies from our laboratory and others concluded that protein tyrosine phosphorylation is one of the early events in activation of monocytes/macrophages by a variety of immune modulators (Manthey, C. L. et al. J. Immunol. (1992) 149:2459) and that protein tyrosine kinase activity is required for activation of monocytes/macrophages for tumoricidal activity and cytokine gene expression (Meisel, C. et al. Eur. J Immunol. (1996) 26:1580). Moreover, activation of monocytes/macrophages by LPS and other immune modulators are associated with activation of multiple proline-directed kinases (Sanghera, J. S. et al. J. Immunol. (1996) 156:4457; Han, J. et al. Science (1994) 265:808; Hambleton, J. et al. J. Exp. Med. (1995) 182:147). These data are consistent with previous findings in monocytes treated by LPS and further demonstrate that similar patterns of intracellular signaling are triggered in cells treated with JBT3002. Specifically, JBT3002 treatment induced tyrosine phosphorylation of proteins with apparent molecule mass of 42 kDa and 38 kDa, caused JNK1 band shift, and induced MAP kinase activation. Additional data show that the 42-kDa and 38-kDa proteins correspond to activated erk and activated p38 MAP kinase, respectively (data not shown).

Activation of monocytes/macrophages by LPS can be significantly facilitated by LBP, a glycoprotein present in the serum (Wright, S. D. et al. Science (1990) 249:1431). LPS binds to LBP and the complex in turn interacts with the LPS receptor CD14, a glycosylphosphatidylinositol-anchored membrane glycoprotein, and triggers many intracellular signaling pathways, such as tyrosine phosphorylation, include stimulation of JNK1 (Hambleton, J. et al. Proc. Natl. Acad. Sci. USA (1996) 93:2774), p38 kinase and MAP kinases (Liu, M. K. et al. J. Immunol. (1994) 153:2642), and translocation of NF-κB (Bellezzo, J. M. et al. Am. J. Physiol. (1996) 270:G956). Moreover, the interaction of the complex with CD14 appears necessary for inducting the expression of a variety of cytokines and inducible nitric oxide synthase by LPS (Gallay, P. et al. J. Immunol. (1993) 150:5086; Sweet, M. J. et al. J. Leukoc. Biol. (1996) 60:8; Stefanova, I. et al. J. Biol. Chem. (1993) 268:20725). Similar results were observed in this study when monocytes were activated by LPS. In sharp contrast, activation of monocytes by JBT3002 appeared not to require LBP or other serum protein. This conclusion is supported by the following findings: (1) induction of TNF-α mRNA expression was increased in the absence of serum; (2) production of TNF-α, IL-1β, and IL-6 induced by JBT3002 was not significantly altered in the absence of serum; and (3) the induction of tyrosine phosphorylation of p42 and p38, band shift of JNK1, and activation of MAP kinases by JBT3002 were not affected by depletion of serum from the culture. Moreover, although CD14-specific monoclonal antibody partially blocked LPS-induced TNF-α production, it did not affect the production stimulated by JBT3002, suggested that the activation of monocytes by JBT3002 was mediated by a receptor unrelated to CD14.

In summary, the new synthetic lipopeptide JBT3002 is a potent activator of tumoricidal properties in human blood monocytes as well as an inducer of cytokine production. JBT3002 triggers several intracellular signaling pathways similar to those stimulated by LPS, but it is independent of LPS binding protein and of CD14 on the monocyte surface.

### EXAMPLE 4

### Evaluation of Oral Administration of MTP-PE and its Tissue-sparing Properties in Combination use with Irinotecan

Irinotecan, a topoisomerase I inhibitor (Camptosar™, CPT-11), is in clinical use for unresectable colon carcinoma and hepatic metastases of this cancer. Side effects include severe myelosuppression and GI tract epithelial toxicity. In the mouse, CT-26P human colon carcinoma injected into the spleen results in rapid growth of liver metastasis in about 3-4 weeks. CPT-11 (ranging from 25-100 mg/kg) causes a dose-dependent reduction in tumor burden of the liver, but rarely any complete eradication of disease. We have observed in our murine model that 100 mg/kg CPT-11 induces loss of structural integrity of duodenal and large colon crypts, including disintegration of villi structure, loss of lamina propria definition and leukocytes and vacuole-filled loss of cytoplasmic structure of epithelia cells of the villi lining. Oral administration of MTP-PE (100 μg/dose) for three consecutive days per week during the two-week regimen of CPT-11 administration (either 4 consecutive ip injections or one injection per week for 4 weeks) to C57BL/6 mice prevents this damage to intestinal tissue. We confirmed this observation for the use of DXR. This protective effect appears to be mediated through cytokine stimulation.

Combination tumoricidal activation of macrophages by oral administration of MTP-PE in combination chemotherapy with CPT-11 appears useful in murine colon carcinoma.

### EXAMPLE 5

### Restoration of Mucosal Integrity: Establishment of Tissue Damage

The purpose of this example is to identify the dose of CPT-11 that causes a defined (and perhaps quantifiable) amount of mucosal damage to intestinal tissue. These findings help define baseline parameters for evaluation of restorative agents to be used with toxic therapies. All studies use C57BL/6 mice.

This protocol used to assess tissue damage after intraperitoneal injection of this drug follows that of Ikuno, N. et. al. (JNCI 87:1876-1883, 1995). The small intestine of each mouse was harvested 4 days after the last injection, according to the following treatment groups:

A. 50 mg/kg once a day for 4 days (i.p.).

B. 75 mg/kg once a day for 4 days (i.p.).

C. 100 mg/kg once a day for 4 days (i.p.).

All harvests of the small intestine were washed in PBS and fix in 10% buffered formalin. Send for routine histology (keep all tissue blocks).

Results: None of the injected mice died. However, mice that received 75 mg or 100 mg treatments displayed clinical signs of toxicity.

### EXAMPLE 6

### Dose-Response Toxicity of CPT-11

Example 5 has shown the protective effect of oral administration of free-form MTP-PE on the subsequent GI tract toxicity of interperitoneal (i.p.) administration of CPT-11 using doses of either 50, 75, or 100 mg/kg. The lethal toxicity of C57BL6 mice to this drug was determined using the following treatment regimens.

Group I. 100 mg/kg CPT-11, i.p., Day 1, Day 2, Day 3, and Day 4.

Group II. 150 mg/kg CPT-11, i.p., Day 1, Day 2, Day 3, and Day 4.

Group III. 200 mg/kg CPT-11, i.p., Day 1, Day 2, Day 3, and Day 4.

Mice were monitored twice a day, with necropsy conducted on moribund mice for small intestine and colon tissue.

| | | | |
|---|---|---|---|
| Results: | Survival: | 5/5 | 100 mg/kg |
| | | 3/5 | 150 mg/kg |
| | | 0/5 | 200 mg/kg |

These data support the 200 mg/kg as the lethal dose.

### EXAMPLE 7

### Prevention of CPT-11 Induced Intestinal Damage by Oral Administration of Free-form MTP-PE

Previous studies have shown that oral administration of free-form MTP-PE (100 μg/dose) given prior to or after chemotherapy with doxorubicin can prevent monocytopenia and loss of mucosal integrity normally observed after treatment with doxorubicin (Oncol. Res. 6:357, 1994). CPT-11 is a topoisomerase inhibitor that induces potent intestinal dysfunction as manifested by loss of structural integrity of intestinal tissue and subsequent loss of mucosal function. Herein, the data show that these effects may be prevented by treatment of mice with MTP-PE before or after administration of CPT-11.

Experimental Design: This study follows our previous experiments in which mice were treated for 3 weeks prior to the administration of doxorubicin (however, it should be noted that the reported effects of CPT-11 are acute, within 4-5 days after multiple injections of CPT-11 mice became sick). Therefor, then endpoints of tissue harvest may not yield optimal evaluation of this form of restorative therapy. This pilot study determines the parameters that may be routinely adjusted in follow-up experiments.

One part of the study evaluated the toxic effect of CPT-11 after pretreatment of the mice for 3 weeks with oral administration of 100 μg/dose of MTP-PE. The mice were treated 3×/week (MTW) for 3 weeks, then CPT-11 was given i.p. as indicated (3 doses) for 4 consecutive days and tissues harvested 3 days later.

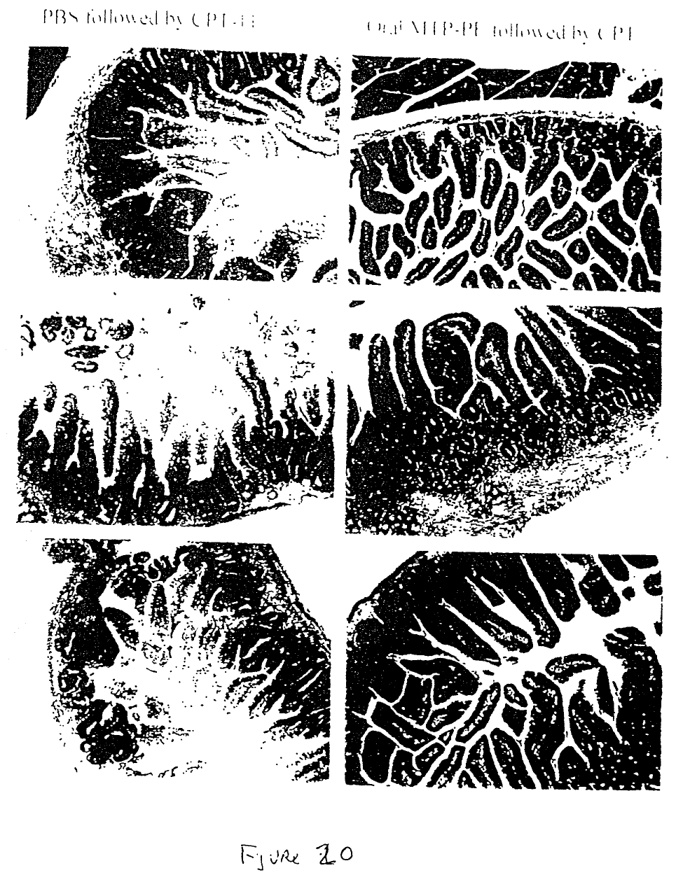

Results: See FIG. 20. Mice receiving oral PBS followed by CPT-11 had severe damage to the intestinal villi and lumen. Mice receiving oral MTP-PE prior to 4 i.p. injections of CPT-11 had no histological (or clinical) evidence of GI toxicity.

### EXAMPLE 8

### Determination of Bioactivity of Oral Administration of JBT3002 to Prevent CPT-11 Induced Intestinal Tissue Damage

This study was designed to measure potential use of JBT3002 as an immunomodulator that can prevent the GI toxicity observed in mice following administration of CPT-11. Mice were given oral administration of different doses of JBT3002 in PC liposomes for 2 weeks (3 consecutive days) prior to i.p. injection of CPT-11 (for 4 consecutive days).

Experimental Design and Methods. Forty (40) C57BL/6 mice (10 mice/group) were fed the PC-JBT3002 liposomes (5 μMol per feeding, 0.2 ml HBSS) for 2 weeks on Day 1, Day 2, an Day 3. After the second set of feedings, the mice were given i.p. injections of CPT-11 (100 mg/kg, 0.2 ml) on Day 1, Day 2, Day 3, and Day 4. Tissue was harvested 7 days after the last injection (small intestine and colon distal to the cecum). Histology was prepared. Some mice were monitored for the presence of drug toxicity.

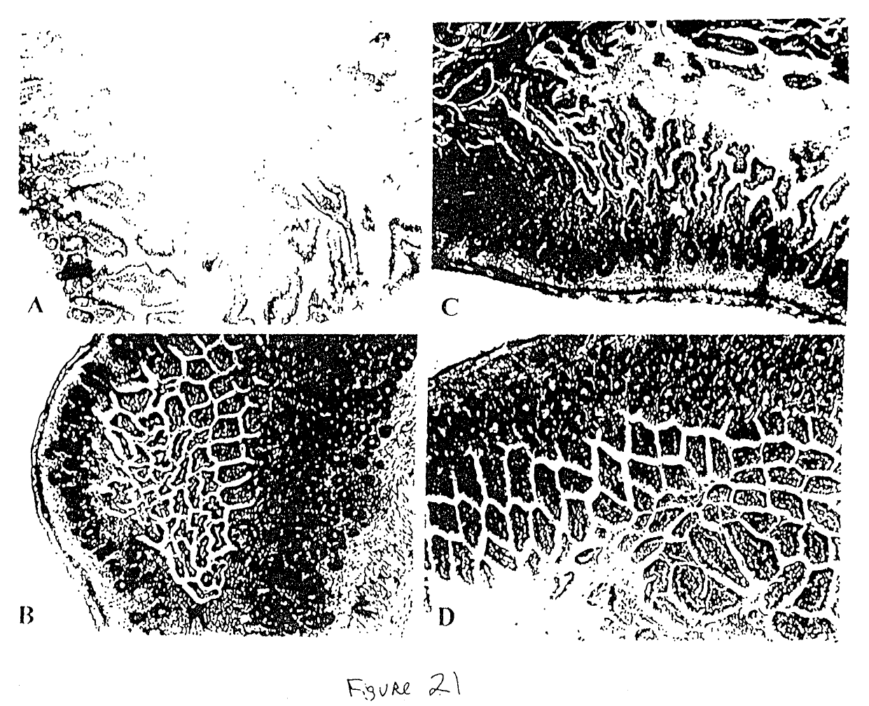

Results: See FIG. 21. Control mice received oral saline (A). Mice received oral JBT3002: 0.1 μg/dose (B); 1.0 μg/dose (C), or 10 μg/dose (D). Note that CPT-11 induced severe toxicity in mice pretreated with saline (A), whereas in mice receiving oral JBT3002 0.1 μg/dose, 1.0 μg/dose, and 10 μg/dose, the intestines were normal (groups B, C, D, respectively).

### EXAMPLE 9

### Determine Minimum Weekly Treatment Schedule

A preliminary study has shown the protective effect of oral administration of free-form MTP-PE on the subsequent GI tract toxicity of i.p. administration of CPT-11 using doses of either 50, 75, or 100 mg/kg. This pilot study used a 3-week prior therapy schedule with 3 feedings, the ongoing study of GI and animal toxicity (CPT-11). The mice were then given i.p. injections of the CPT-11 at 100 mg/kg for 4 consecutive days and tissue harvest to take place 3 and 10 days following the last per week of free-form MTP-PE.

Experimental Design. Groups of 10 C57BL/6 mice received oral feedings of 100 μg/dose of free-form MTP-PE for either one, two, or three consecutive weeks prior to injection with CPT-11 at a dose to be determined by drug injection.

Results: Three oral administrations of MTP-PE (1 week) were sufficient to prevent GI toxicity by CPT-11 (even at 100 mg/kg).

### EXAMPLE 10

### Determine Ability to Prevent Morbidity

Example 5 has shown the protective effect of oral administration of free-form MTP-PE on the subsequent GI tract of i.p. administration of CPT-11 using doses of either 50, 75, or 100 mg/kg. Example 6 has shown that 100, 150, and 200 mg/kg at 4 consecutive i.p. administrations are highly toxic to C57BL/6 mice. This Example demonstrates the protection of mice against the toxicity of CPT-l 1 by pretreatment of the mice with 2 consecutive weeks of 100 μg/dose MTP-PE prior to administration of the drug. See Table 6.

TABLE 6
Experimental Design: Groups of 10 C57BL/6 mice
will receive oral feedings of 100 ug/dose of
free-form MTP-PE for one week prior to 4 daily i.p.
injections with CPT-11 at 100,150, or 200
mg/kg

| Results: | Oral therapy | CPT-11 | Death | Morbidity |
|---|---|---|---|---|
| | Saline | 100 mg/kg | 0/10 | 8/10 |
| | Saline | 150 mg/kg | 6/10 | 10/10 |
| | Saline | 200 mg/kg | 9/10 | 10/10 |
| | MTP-PE | 100 mg/kg | 0/10 | 0/0 |
| | MTP-PE | 150 mg/kg | 2/10 | 4/10 |
| | MTP-PE | 200 mg/kg | 6/10 | 8/10 |

### EXAMPLE 11

### JBT3002 Series: Combination Therapy of CT-26P Murine Colon Carcinoma in Balb/c Mice with CPT-11 & Oral Administration of JBT3002 Encapsulated into Liposomes

Purpose: This Example demonstrates the ability of different doses of CPT-11 to inhibit the growth of CT-26P colon carcinoma in the liver of mice and whether the therapeutic efficacy of this drug can be enhanced by the oral administration of the macrophage activator, JBT3002.

Experimental design: Mice are given an intrasplenic injection of 15,000 cultured CT-26P cells on day 0 and then receive either no further therapy, 3 oral feedings of 1 μg JBT3002 in PC liposomes per week, one ip injection of CPT-11 per week, or the combination of the CPT-11 plus oral administration of JBT3002. This course of therapy was repeated weekly for about three weeks prior to tissue harvest. See Table 7.

Experimental Groups/Procedures: 40 Balb/c mice were divided into 8 groups of 5 mice each. On day 0, the spleens of the mice were injected with 15,000 cultured CT-26P cells. The groups were then treated as follows:

| | | Days of the Week (for three weeks) | | | | | | | | | | | | |
|---|---|---|---|---|---|---|---|---|---|---|---|---|---|---|
| | | T | W | R | F | M | W | R | F | M | W | R | F | M |
| I. | No therapy | TC | — | — | — | — | — | — | — | — | — | — | — | — |
| II. | CPT-11 (25 mg/kg) | | — | — | — | C | — | — | — | C | — | — | — | C |
| III. | CPT-11 (50 mg/kg) | | — | — | — | C | — | — | — | C | — | — | — | C |
| IV. | CPT-11 (100 mg/kg) | | — | — | — | C | — | — | — | C | — | — | — | C |
| V. | JBT3002 (1 mg/dose) | | X | X | X | — | X | X | X | — | X | X | X | — |
| VI. | CPT-11 (25) + JBT3002 | | X | X | X | C | X | X | X | C | X | X | X | C |
| VII. | CPT-11 (50) + JBT3002 | | X | X | X | C | X | X | X | C | X | X | X | C |
| VIII. | CPT-11 (100) + JBT3002 | | X | X | X | C | X | X | X | C | X | X | X | C |

Following the third ip injection of CPT-11, the mice were closely monitored for symptoms of extensive growth of tumor in the liver. On the day of harvest, the following tissues were prepared:

1. Weigh spleens (see Table 8)

2. Weigh livers (see Table 9)

3. The “grade” of liver tumor (0-no tumor; I-<5 small mets; II-5-20 mets, III=>20 mets) was determined.

4. The small intestine and large colon were harvested for histology (H&E).

The tissue were placed on “end” orientation in order to visualize the villi of the intestine.

The spleens of Balb/c mice were injected with 15,000 cultured CT-26P colon carcinoma cells on day 0. Mice received no further treatment (controls) or treatment with CPT-11 (at 25, 50 or 100 mg/kg) once a week starting on day 7, or oral administration of JBT3002 beginning on day 1 (1 μg/dose) and continuing three times per week. Therapy was discontinued on day 17 due to the health of the control mice. Treated mice received two weeks of chemotherapy and three weeks of the macrophage activator. Tissues were harvested on day 17. The spleens and livers were weighed, the extent of metastasis was graded (denoted below) and histology prepared.

TABLE 8
Therapy of CT-26P Murine Colon Carcinoma in Syngeneic Balb/c
Mice with Combination Chemotherapy and Oral Administration
of Liposome-encapsulated JBT3002
Spleen Weights (grams)

| Control | CPT-11 (25) | CPT-11 (50) | CPT-11 (100) | JBT3002 |
|---|---|---|---|---|
| 1.301 | 1.403 | 0.419 | 0.252 | 0.148 |
| 1.385 | 1.486 | 0.151 | 0.120 | 1.048 |
| 1.154 | 0.888 | 0.459 | 0.171 | 1.412 |
| 1.399 | 1.056 | 0.894 | 0.122 | 0.849 |
| 0.889 | 1.588 | 1.750 | 0.414 | 1.227 |
| 1.225 ± 0.189 | 1.284 ± 0.257 | 0.746 ± 0.561* | 0.216 ± 0.110* | 1.137 ± 0.628 |

| CPT-11 (25) + JBT3002 | CPT-11 (50) + JBT3002 | CPT-11 (100) + JBT3002 |
|---|---|---|
| 0.158 | 0.918 | 0.216 |
| 1.183 | 0.755 | 0.380 |
| 0.396 | 0.508 | 0.237 |
| 1.369 | 0.563 | 0.609 |
| 0.512 | 0.491 | 0.125 |
| | 0.302 | |
| 0.723 ± 0.468** | 0.589 ± 0.198* | 0.313 ± 0.169* |

*significant reduction in spleen tumor burden compared to control, p < 0.05
**significant reduction in spleen tumor burden compared to both control and the use of CPT-11 (25 mg/kg) only, p < 0.05

TABLE 9
Therapy of CT-26P Murine Colon Carcinoma in Syngeneic Balb/c
Mice with Combination Chemotherapy and Oral Administration
of Liposome-encapsulated JBT3002
Liver Weights, grams (tumor grade)

| Control | CPT-11 (25) | CPT-11 (50) | CPT-11 (100) | JBT3002 |
|---|---|---|---|---|
| 1.311 (1) | 2.246 (2) | 1.453 (0) | 1.196 (1) | 1.126 (1) |
| 1.522 (1) | 1.416 (1) | 1.407 (1) | 1.105 (0) | 1.901 (3) |
| 1.465 (2) | 1.680 (2) | 1.205 (2) | 1.252 (1) | 2.421 (3) |
| 1.876 (3) | 1.460 (2) | 1.636 (0) | 1.172 (0) | 1.443 (1) |
| 1.520 (1) | 1.549 (0) | 1.525 (2) | 1.293 (1) | 2.230 (1) |
| 1.539 ± 0.188 | 1.670 ± 0.301 | 1.445 ± 0.142 | 1.203 ± 0.065 | 1.842 ± 0.498 |

| CPT-11 (25) + JBT3002 | CPT-11 (50) + JBT3002 | CPT-11 (100) + JBT3002 |
|---|---|---|
| 1.071 (0) | 1.649 (1) | 1.219 (0) |
| 1.476 (0) | 1.528 (1)* | 1.336 (0) |
| 1.399 (0) | 1.262 (0) | 1.347 (1)** |
| 1.222 (1) | 1.387 (1) | 1.205 (0) |
| 1.336 (0) | 1.346 (1) | 0.916 (0) |
| | 1.088 (1) | |
| 1.301 ± 0.142 | 1.377 ± 0.180 | 1.204 ± 0.155 |

*1 tumor nodule
**2 tumor nodules
Grade: 0-no visible tumor; -<5 mets; 2 = 5-20 mets; 3 = > 20 mets

### EXAMPLE 12

### Therapy of Cancer Metastasis by CPT-11 and Oral Administration of JBT3002

JBT3002 was formulated without encapsulation into liposomes. The JBT3002 compound was added to Hank's balanced salt solution (HBSS) at 1-3 mg/ml and sonicated at ice temperature for 20 min (80 kilocycles, 80 watts). This results in a slightly opaque solution that, when diluted further, immediately is soluble in HBSS. This stock solution is stored at 4 C. (made fresh every 4 weeks) and is diluted into HBSS for oral feedings (e.g., 0.25 μg/ml) which is a clear solution.

BALB/c mice were injected into the spleen with viable syngeneic CT-26 colon carcinoma cells. Groups of mice were treated or gauged with different doses of JBT3002 once daily for 3 consecutive days. Two days after the third oral gauge with JBT3002, the mice were injected i.p. with 100 mg/kg CPT-11. In one series of studies, the mice received CPT-11 once/day for 4 consecutive days (intensive regimen). In a second series of studies, the mice received injections of CPT-11 once/week for 3 consecutive weeks (chronic regimens). JBT3002 was always administered for 3 consecutive days prior to CPT-11. The mice were killed at different time points after the last cycle of therapy. Spleen tumor and liver metastases (experimental) were quantified.

The results are presented in tabular form in Tables 10-16.

Table 10. This study compared the efficacy of liposome-encapsulated JBT 3002 and JBT 3002 (sonicated drug). The data show the effectiveness of sonicated free-form JBT 3002.

Table 11. The purpose of this study was to determine a dose dependence for efficacy of JBT 3002 9free-form). Note that 0.01 μg/dose of JBT 3002 (sonicated) produced similar therapeutic effects as 1.0 μg/dose of MLV-JBT 3002. The data show the effectiveness of sonicated free-form JBT 30002.

Table 12. In this study, CPT-11 was administered under intensive schedule. Again, free-form JBT 3002 was more potent than liposomal formulation of JBT 3002.

Table 13. Dose response of sonicated (free-form) JBT 3002 showing that optimal dose is 0.01-0.001 μg/dose.

Table 14. The data confirm that free-form JBT 3002is more potent than liposomal formulation.

Table 15. CPT-11 alone inhibited liver metastases, but 7 of 10 mice died during therapy. JBT 3002 produced a reduction in liver metastases. The combination of oral JBT 3002 (free form) and intensive CPT-11 (i.p.) produced significant therapy of liver metastases in all mice without any side effects.

Table 16. The clinical course of therapy (CPT-11 once weekly) combined with oral JBT 3002 (0.05 μg/dose) produced significant inhibition of liver metastasis.

### EXAMPLE 13

### Preparation of Tablet to Free-form JBT3002

JBT 3002 was formulated in tablets (100 μg/tablet): croscarmellose sodium, NF (8.0 μm); lactose anhydrous, NF (299.9 gm); microcryst. Cellulose, type pH-102, NF (80.0 gm), silicon dioxide, colloidal, NF (8.0 gm), magnesium stearate, NF (4.0 gm), JBG 3002 (0.10 gm), distilled water (30.0 gm).

Preparation of tablet to free-form in water. Tablet placed in 10 ml sterile, pyrogen-free water for 2-3 min with shaking at room temperature, then 10 ml more water added using Lot PCOO2.115. At 100 μg/tablet, this gave 5 ug/ml active material. Solution opaque with “carrier”-appearing material. Settled to bottom. Placed at 37° C. for about 5 min. pH tested at 8.0. Two aliquots of 10 ml taken. One aliquot was taken to pH 1.5 by a single drop of concentrated HCl. Both samples were placed in a water bath at 37° C. for 30 min. The low pH sample was restored to pH 7.7 with NaOH (took about 5 drops of the NaOH, IN solution). The samples were brought to 50 ml by addition of water, and the final pH was 8.0 for the original and 7.43 for the low pH sample. The solutions were still slightly opaque and filtering with a 0.2 micron filter cleared the solution. Aliquots of about 25 ml were measured in an assay for NO production by macrophages and testing of the working dilution for endotoxin.

Table 17. The data show that JBT 3002 in tablets is biologically active and is resistant to pH 1.5 (30 min).

Table 18. The data confirm that JBT 3002 in tablets is biologically active and suffers no loss of potency.

Table 19 (A, B, C). Therapy of human pancreatic carcinoma liver metastasis. The pancreas of nude mice were injected with 1×10⁶ viable human pancreatic cancer L3.6pl cells. JBT 3002 (tablets) were given by gauge (0.05 μg/dose) for 3 consecutive days followed by i.p. injection of CPT-11 (100 mg/kg). This regimen was repeated on a weekly basis for 3 weeks. The mice were killed. Pancreatic tumors, liver metastases, and lymph node metastasis were quantified. The data in Table 19 show that oral JBT 3002 plus CPT-11 resulted in effective therapy: This combination resulted in the inhibition of both liver metastases and lymph node metastases.

Table 20, Table 21. Ongoing studies to evaluate the therapeutic efficacy of JBT 3002 tablets plus CPT-11 against human colon cancer (Table 20) and mouse colon cancer (Table 21) liver metastasis.

### EXAMPLE 14

### Protection from CPT-11-Induced Intestinal Toxicity by Oral Administration of JBT 3002: Induction of IL-15 in the Lamina Propria of the Intestine Background

The Interleukin-15 (IL-1 5) binds to the common γ c and the IL-2 receptor β subunit for signal transduction (Grabstein, K. H., et al., Science (1994) 264:965-968; Carson, W. E., et al., J Exp Med (1994) 180:1395-1403; Giri, J. G., et al., EMBO J (1994) 13:2822-2830). IL-15 shares many of the biological activities of IL-2, including: generation of CTL and LAK cells (Grabstein, K. H., supra.); activation of NK cells to produce IFN-γ, TNF-α, and GM-CSF (Carson, W. E., supra.); B cell proliferation and differentiation (Armitage, R. J., et al., J Immunol (1995) 154:483-490).

IL-15 is expressed in a variety of tissues, including: placenta; skeletal muscle; kidney; liver; IFN-γ/LPS-activated macrophages (Doherty, T. M., et al., J Immunol (1996) 156:735-741), but not activated T cells (Grabstein, K. H., supra).

The expression of IL-15 (Reinecker, H. C., et al., Gastroenterology (1996) 111:1706-1713) has been shown from isolated rat intestinal epithelial cells, which constitutively express IL-15. IEC-6 cells express IL-15, as well as isolated human intestinal epithelial cells; Lamina propria mononuclear cells; several human intestinal epithelial tumor-derived cells lines, including Caco-2 and HT29. IL-15 mRNA expression in Caco-2 cells has been shown to be upregulated by IFN-γ.

Further, intestinal epithelial cell lines and primary intestinal epithelial cells express “intermediate affinity receptors” for IL-2, which is composed of the common γ c and the IL-2 receptor β subunit (Reinecker, H. C., et al., Proc Natl Acad Sci USA (1995) 92:8353-8357; Ciacci, C., et al., J Clin Invest (1993) 92:527-532). Upon incubation with recombinant IL-15 (rIL-15), it has been observed that there is a stimulation of protein tyrosine phosphorylation in Caco-2 cells (Reinecker, H. C., supra, 1996). Also, rIL-15 can stimulate the proliferation of Caco-2 cells as determined by [³H]thymidine uptake (Reinecker, H. C., supra, 1996).

Administration (i.p.) of rIL-15 has demonstrated some protection against chemotherapy-induced intestinal toxicity in a rat model: 5-FU (Cao, S., et al., Cancer Res (1998) 58:1695-1699) and irinotecan (Cao, S., et al., Cancer Res (1998) 58:3270-3274).

### INTRODUCTION

Recent data published by Cao et al. (supra) show that multiple injections of IL-15 can protect against toxicity medicated by 5-FU or irinotecan in a preclinical animal model. Whether the mechanism by which oral administration of JBT 3002 protected against toxicity mediated by CPT-11 was via upregulation of IL-15 in the intestines was evaluated.

### RESULTS

BALB/c mice were injected with CT-26 colon cancer cells into the spleen. Treatment with CPT-11 alone (100 mg/kg), JBT 3002 alone (0.05 μg/dose), or JBT 3002 (0.05 ag/dose) followed by CPT-11(100 mg/kg) was carried out as described previously. Four days after the last injection of CPT-11, some mice were killed and their ileum was harvested and prepared for histology, immunohistochemistry, and molecular biology analyses.

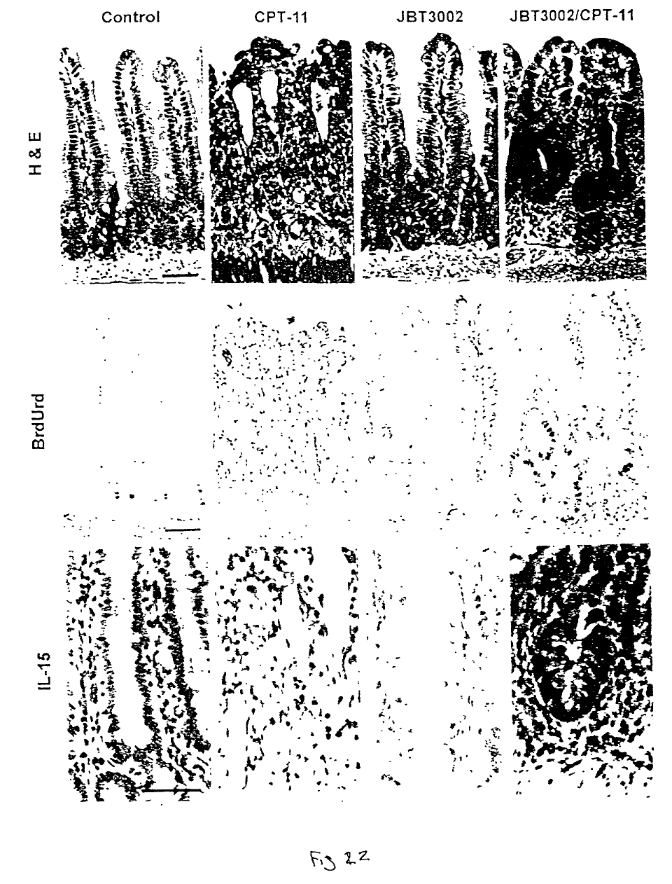

The data shown in FIG. 22 (ileum) demonstrate the following: administration of CPT-11 alone produces disruption of the intestinal architecture (H&E). Treatment with JBT 3002 and CPT-11 prevents this pathology. These data confirm our earlier results. Immunohistochemistry for BrdUrd (cell division) shows that in mice given JBT 3002 plus CPT-11, there is an increased number of dividing epithelial cells (BrdUrd).

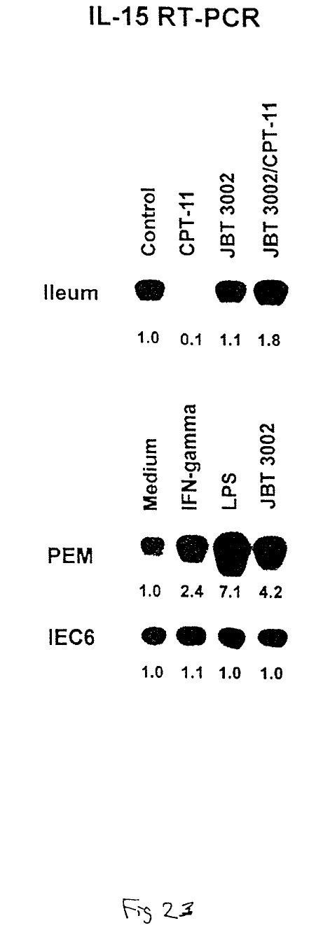

Immunostaining for IL-15 shows that the lamina propria of ileum from mice receiving JBT 3002 (oral) and CPT-11 (i.p.) has high expression for IL-1 5. To determine whether macrophages or epithelial cells respond to JBT 3002 by upregulating IL-1 5, the RT-PCR technique was used and the results are shown in FIG. 23.

CPT-11 produces a significant decrease in expression of IL-15 in the ileum. JBT 3002 restores or augments this expression.

Peritoneal exudate macrophages (PEM), but not intestinal cells (IEC6), upregulate IL-15 expression in response to JBT 3002.

CT-26 tumor lesions in the liver of BALB/c mice was also studied. The number of macrophages (Scav-R-positive) producing nitric oxide (iNOS) and IL-15 is clearly increased in regressing metastases of mice treated with both JBT 3002 and CPT-11.

### CONCLUSIONS

The oral administration of JBT 3002 upregulates expression of IL-15 in intestinal macrophages. The production of endogenous IL-15 protects the intestine against toxicity mediated by CPT-11.

Each of these publications is hereby incorporated herein by reference. Said publications relate to the art to which this invention pertains. The references cited above are each incorporated by reference herein, whether specifically incorporated or not.

TABLE 1
Production of nitric oxide and tumoricidal properties in mouse macrophages
by liposomes containing MTP-PE, CGP31362 and JT3002

| Concentration | MLV-HBSS | | MLV-MTP-PE | | MLV-31362 | | MLV-JT3002 | |
|---|---|---|---|---|---|---|---|---|
| of MLV | NO | Cytotoxicity | NO | Cytotoxocity | NO | Cytotoxicity | NO | Cytotoxicity |
| (nmol/well) | (μM) | (%) | (μM) | (%) | (μM) | (%) | (μM) | (%) |
| 50 | 8 | 4 | 4 | 19 | 28^{a} | 84^{a} | 30^{a} | 86^{a} |
| 25 | 5 | 0 | 2 | 14 | 26^{a} | 74^{a} | 29^{a} | 80^{a} |
| 12 | 1 | 1 | 2 | 10 | 23^{a} | 79^{a} | 23^{a} | 84^{a} |
| 6 | 1 | 2 | 2 | 5 | 22^{a} | 72^{a} | 22^{a} | 70^{a} |
| 3 | 1 | 0 | 2 | 4 | 20^{a} | 75^{a} | 22^{a} | 68^{a} |
| Macrophages (1 × 10⁵/well) were incubated with the indicated concentrations of MLV in medium containing 10 U/ml IFN-γ. All MLV contained 1 mg immunomodulator/300 μM phospholipids. NO production (nitrite/nitrate) was determined one day later. The cultures were washed and 1 × 10⁴ [³H]TdR-labeled A375P cells were added. Assays were terminated 72 h later. Macrophages incubated in medium alone (negative control) produced 0.2 μM NO and | | | | | | | | |
| # 10% cytotoxicity. Macrophages in medium containing LPS (1 μg/ml) and IFN-γ (10 U/ml) produced 26 μM NO and 48% cytotoxicity (P < 0.001). The values are the mean of triplicate cultures. Variation from the mean did not exceed 10%. These are the results of one representative experiment of four. | | | | | | | | |

^{a}P < 0.001.

TABLE 2
Minimal concentration of liposome-JT3002 required to
induce production of nitric oxide in murine macrophages

| Lipid | NO (μM | | | |
|---|---|---|---|---|
| concentration | JT3002 | JT3002 | JT3002 | JT3002 |
| (nmol/well) | (0.1 mg) | (0.02 mg) | (0.004 mg) | (0.0008 mg) |
| 25 | 27^{a} | 23^{a} | 10^{a} | 11 |
| 12.5 | 26^{a} | 20^{a} | 14^{a} | 9 |
| 6.2 | 24^{a} | 17^{a} | 12^{a} | 7 |
| 3.1 | 24^{a} | 16^{a} | 10 | 7 |
| 1.6 | 21^{a} | 13^{a} | 9 | 7 |
| 0.8 | 17^{a} | 11 | 9 | 7 |
| 0.4 | 19^{a} | 11 | 10 | 7 |
| 0.2 | 18^{a} | 10 | 10 | 6 |
| Macrophages (1 × 10⁵/well) were incubated in medium containing 10 U/ml IFN-γ (control) or medium containing 10 U/ml IFN-γ and different concentrations of liposomes containing 0.1 mg, 0.02 mg, 0.004 mg, or 0.008 mg JT3002 in 300 μM phospholipids. NO production was determined 24 h later. The values are the mean NO proudction in μM of triplicate cultures. | | | | |
| # Variation from the mean did not exceed 10%. Macrophages incubated with medium plus IFN-γ or medium containing | | | | |
| # IFN-γ plus LPS produced 9 and 25 μM NO, respectively. This is one representative experiment of three. | | | | |

^{a}P < 0.001.

TABLE 3
Activation of tumoricidal properties in macrophages from iNOS knockout mice

| Lipid | | | | | | |
|---|---|---|---|---|---|---|
| concentration | NO (μM) | | | Cytotoxicity (%) | | |
| (nmol/well) | +/+ mice | +/− mice | −/− mice | +/+ mice | +/− mice | −/− mice |
| 50 | 21^{a} | 14^{b} | 0 | 93^{a} | 91^{a} | 7 |
| 25 | 20^{a} | 14^{b} | 0 | 93^{a} | 89^{a} | 1.5 |
| 10 | 17^{a} | 12 | 0 | 85^{a} | 62^{a} | 0 |
| 5 | 16^{a} | 11 | 0 | 31^{a} | 51^{a} | 0 |
| 0 | 0 | 0 | 0 | 0 | 0 | 0 |
| LPS (1 μg/ml) | 20^{a} | 13 | 0 | | | |
| Macrophages (1 × 10⁵/well) were incubated in medium containing 10 U/ml IFN-γ (control) or medium containing 1 μg/ml LPS (positive control), or medium containing different concentrations of MLV containing 0.1 mg JT3002/300 μM phospholipid. After 20 h incubation, the cultures were washed and 1 × 10⁴ [³H]TdR-labeled K-1735 M2 (shown) or CT-26 (not shown) cells were added. NO production (μM/10⁴ macrophages) was determined after 20 h and | | | | | | |
| # cytotoxicity was determined after 72 h. The values are the mean of triplicate samples. Variation from the mean did not exceed 15%. This is one representative experiment of three. | | | | | | |

^{a}P < 0.01.
^{b}P < 0.05.

TABLE 4
Activation of tumoricidal properties in macrophages from
LPS-responsive (C3H/HeN) and -nonresponsive (C3H/HeJ) mice

| Lipid | C3H/HeN mice | | C3H/HeJ mice | |
|---|---|---|---|---|
| concentration | NO | | NO | |
| (nmol/well) | (μM) | Cytotoxicity (%) | (μM) | Cytotoxicity (%) |
| 20 | 23^{a} | 35^{a} | 32^{a} | 40^{a} |
| 2 | 11 | 28^{a} | 26^{a} | 32^{a} |
| 0.2 | 2 | 13 | 13 | 27^{a} |
| 0.02 | 5 | 7 | 9 | 11 |
| 0 | 2 | 3 | 0 | 6 |
| LPS (1 μg/ml) | 23^{a} | 36^{a} | 8 | 12 |
| Macrophages (1 × 10⁵/well) were incubated in medium containing 10 U/ml IFN-γ (control), or medium containing 1 μg/ml LPS (positive control), or medium containing different concentrations of MLV containing 0.1 mg JT3002/300 μM phospholipid. After 20 h incubation, the cultures were washed and 1 × 10⁴ [³H]TdR-labeled K-1735 M2 cells were added. NO production (nitrite) was determined after 20 h and cytotoxicity was determined after 72 h. | | | | |
| #The values are the mean of triplicate samples. Variation from the mean did not exceed 10%. This is one representative experiment of three. | | | | |

^{a}P < 0.01.

TABLE 5
Duration of tumoricidal activity in macrophages
incubated with liposomes containing JT3002

| Days post- | NO (μM) | | Cytotoxicity (%) | |
|---|---|---|---|---|
| activation | Medium | JT3002 | Medium | JT3002 |
| 1 | 0.9 | 31.8^{a} | 5.9 | 49.7^{a} |
| 2 | 1.3 | 34.0^{a} | 6.6 | 19.8^{b} |
| 3 | 0.7 | 27.7^{a} | 4.1 | 19.2^{b} |
| 4 | 4.9 | 4.0 | 5.9 | 4.8 |
| Reactivation | | | | |
| 5 | 2.2 | 33.7^{a} | 3.0 | 41.0^{a} |
| Macrophages (1 × 10⁵/well) were incubated in medium containing 10 U/ml IFN-γ (control) or medium containing 10 U/ml IFN-γ plus 1 nmol/well of MLV containing 0.1 mg JT3002/300 μM phospholipid. After 20 h incubation, the cultures were washed and fresh medium was added for 0, 1, 2, 3, or 4 days. At the different time points, 1 × 10⁴ [³H]TdR-labeled CT-26 cells were added. | | | | |
| # NO production (nitrite/nitrate) was determined at the indicated times. Cytotoxicity was determined after 72 h of continuous tumor-cell-macrophage interaction. The values are the mean of triplicate cultures. Variation from the mean did not exceed 10%. This is one representative experiment of two. | | | | |

^{a}P < 0.001.
^{b}P < 0.01.

TABLE 7
Combination Therapy of MTP-PE and CPT-11 for Mouse
CT-26 Colon Cancer Liver Metastasis

| | | Spleen | | Liver | |
|---|---|---|---|---|---|
| Oral | | Weight | Tumor | | Median no. |
| treatment | CPT- 11 | (g) | size (mm) | Weight | metastases |
| Saline | Saline | 1.5 ± 0.1 | 1.4 ± 0.7 | 7.4 ± 1.6 | >100 |
| Saline | 50 mg/kg | 0.6 ± 0.2 | 8.3 ± 2.0 | 2.0 ± 0.3 | 30 |
| Saline | 100 mg/kg | ------------------All mice died.------------------------ | | | |
| MTP-PE | 50 mg/kg | 0.6 ± 0.2 | 10.4 ± 2 | 2.2 ± 0.7 | 30 |
| MTP-PE | 100 mg/kg | 0.3 ± 0.1 | 5.6 ± 2 | 1.2 ± 0.1 | 4 |

TABLE 10
Therapy of experimental liver metastasis produced by murine CT-26 colon carcinoma with CPT-11 in combination
with either MLV-JBT 3002 or free-form (FF) JBT 3002

| | | Spleen (primary) | | Liver (metastasis) | | |
|---|---|---|---|---|---|---|
| | ΔBW^{a} | | Tumor volume | | | Liver weight |
| Treatment | (%) | Incidence | (mm³) | Incidence | Median (range) | (g) |
| MLV-HBSS | 6.4 | 5/5 | 567 ± 94 | 5/5 | 46, 56, 72, >100, >100 | 3.5 ± 1.6 |
| MLV-HBSS + CPT-11 | −1.7 | 5/5 | 140 ± 30^{c} | 5/5 | 12, 15, 18, 39, 73 | 1.8 ± 0.3^{b} |
| MLV-JBT3002 (1.0 μg/dose) + CPT-11 | −0.4 | 5/5 | 56 ± 29^{c} | 2/5 | 0, 0, 0, 6, 12 | 1.6 ± 0.2^{b} |
| MLV-JBT3002 (0.1 μg/dose) + CPT-11 | −0.8 | 5/5 | 72 ± 15^{c} | 3/5 | 0, 0, 4, 8, 79 | 1.6 ± 0.2^{b} |
| FF-JBT3002 (1.0 μg/dose) + CPT-11 | −3.9 | 5/5 | 202 ± 69^{b} | 5/5 | 7, 25, 37, 53, 81 | 1.8 ± 0.4^{b} |
| FF-JBT3002 (0.1 μg/dose) + CPT-11 | 0 | 5/5 | 85 ± 23^{c} | 3/5 | 0, 0, 9, 13, 35 | 1.5 ± 0.3^{b} |

Five BALB/c mice per group were given intrasplenic injection of 1 × 10⁴ CT-26 cells on day 0. Mice were treated with repeated oral feedings of
MLV-JBT3002 (at either 1.0 ot 0.1 μg/dose, 5 μmol PCPS MLV), or FF-JBT3002 (at either 1.0 or 0.1 μg/dose) thrice weekly for 3 weeks beginning 3
days after tumor cell inoculation, in combination with 100 mg/kg CPR-11 i.p. once a week (on day 7, 14, and 21). All groups were killed on day 23.

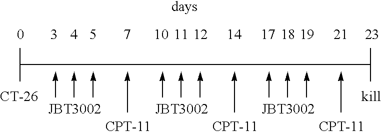

^{a}The rate of body weight reduction was calculated with the formula ΔBW (%) = (A B − 1) × 100, where A = mean body weights of mice at
death, and B = mean body weights of mice on day 0.
^{b}P < 0.05,
^{c}P < 0.005, compared with MLV-HBSS

TABLE 11
Therapy of experimental liver metastasis produced by murine CT-26 colon carcinoma with CPT-11 in combination
with either MLV-JBT 3002 or free-form (FF) JBT 3002

| | | Spleen (primary) | | Liver (metastasis) | | |
|---|---|---|---|---|---|---|
| | ΔBW^{a} | | Tumor volume | | | Liver weight |
| Treatment | (%) | Incidence | (mm³) | Incidence | Median (range) | (g) |
| MLV-HBSS + saline | 2.4 | 5/5 | 701 ± 268 | 5/5 | 54, >100, >100, >100, | 4.2 ± 1.2 |
| | | | | | >100 | |
| CPT-11 | −1.5 | 5/5 | 189 ± 71^{c} | 5/5 | 22, 34, 39, 47, 57 | 1.7 ± 0.3^{c} |
| MLV-JBT3002 (1.0 μg/dose) + CPT-11 | −1.4 | 5/5 | 154 ± 136^{c} | 3/5 | 0, 0, 3, 4, 13 | 1.4 ± 0.1^{c} |
| FF-JBT3002 (1.0 μg/dose) + CPT-11 | 0 | 5/5 | 238 ± 70^{b} | 5/5 | 5, 27, 31, 53, 80 | 1.7 ± 0.4^{c} |
| FF-JBT3002 (0.1 μg/dose) + CPT-11 | 1.7 | 5/5 | 290 ± 106^{b} | 5/5 | 1, 3, 10, 14, 34 | 1.5 ± 0.5^{c} |
| FF-JBT3002 (0.01 μg/dose) + CPT-11 | −1.0 | 5/5 | 181 ± 115^{c} | 4/5 | 0, 1, 3, 14, 32 | 1.4 ± 0.4^{c} |

BALB/c mice were given intrasplenic injection of 1 × 10⁴ CT-26 cells on day 0. Mice were treated with oral feedings of MLV-JBT3002 (at either 1.0 or
0.1 μg/dose, 5 μmol PCPS MLV), or FF-JBT3002 (at either 1.0 or 0.1 μg/dose) thrice weekly for 3 weeks beginning 3 days after tumor cell inoculation,
in combination with 100 mg/kg CPT-11 i.p. once a week (on day 7, 14, and 21). All groups were killed on day 24.

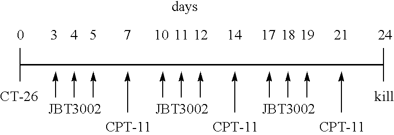

^{a}Changes in body weight were calculated by the formula ΔBW (%) = (A − B) × 100, where A = mean body weight of mice at death, and B = mean body
weight of mice on day 0
^{b}P < 0.05,
^{c}P < 0.005, compared with MLV-HBSS + saline

TABLE 12
Therapy of experimental liver metastasis produced by murine CT-26 colon carcinoma with intensive CPT-11
injections in combinations with either MLV-JBT 3002 or free-form (FF) JBT 3002 at different doses

| | | Spleen (primary) | | Liver (metastasis) | | |
|---|---|---|---|---|---|---|
| | ΔBW^{a} | | Tumor volume | | | Liver weight |
| Treatment | (%) | Incidence | (mm³) | Incidence | no. | (g) |
| MLV-HBSS + saline | 5.1 | 5/5 | 153 ± 62 | 5/5 | 23, 26, 71, >100, >100 | 2.4 ± 1.0 |
| MLV-HBSS + CPT-11 | −17.6 | 5/5 | 52 ± 30 | 2/5 | 0, 0, 0, 1, 6 | 1.2 ± 0.1 |
| MLV-JBT3002 (1.0 μg/dose) + CPT-11 | −1.5 | 5/5 | 45 ± 10 | 0/5 | all 0 | 1.4 ± 0.1 |
| FF-JBT3002 (1.0 μg/dose) + CPT-11 | −2.4 | 5/5 | 48 ± 8 | 2/5 | 0, 0, 0, 3, 5 | 1.4 ± 0.03 |
| FF-JBT3002 (0.1 μg/dose) + CPT-11 | −2.2 | 5/5 | 50 ± 16 | 1/5 | 0, 0, 0, 0, 3 | 1.4 ± 0.2 |
| FF-JBT3002 (0.01 μg/dose) + CPT-11 | 0.4 | 5/5 | 29 ± 26 | 4/5 | 0, 2, 2, 26, 27 | 1.6 ± 0.1 |
| FF-JBT3002 (0.001 μg/dose) + CPT-11 | −6.9 | 5/5 | 56 ± 25 | 1/5 | 0, 0, 0, 0, 3 | 1.4 ± 0.2 |
| FF-JBT3002 (0.0001 μg/dose) + CPT-11 | −15.4 | 5/5 | 28 ± 20 | 3/5 | 0, 0, 1, 2, 5 | 1.1 ± 0.1 |

BALB/c mice were injected into the spleen with 1 × 10⁴ viable CT-26 cells on day 0. Mice were treated with oral feedings of 5 μmol MLV-HBSS,
MLV-JBT3002 (1 μg/dose), or FF-JBT3002 (at either 1.0, 0.1, 0.001, or 0.0001 μg/dose) for 3 consecutive days beginning 3 days after tumor cell
inoculation. Seven days later, groups of mice received 4 daily i.p. injections of 100 mg/kg CPT-11. All groups were killed on day 14.

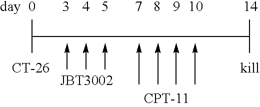

^{a}Changes in body weight were calculated by the formula ΔBW (%) = (A − B)B × 100, where A = mean body weight of mice at death, and B = mean
body weight of mice on day 0.

TABLE 13
Therapy of experimental liver metastasis produced by murine CT-26 colon carcinomas with once weekly CPT-11
injections in combination with either MLV-JBT 3002 or free-form (FF) JBT 3002 at different doses

| | | Spleen (primary) | | Liver (metastasis) | | |
|---|---|---|---|---|---|---|
| | ΔBW^{a} | | Tumor volume | | | Liver weight |
| Treatment | (%) | Incidence | (mm³) | Incidence | no. | (g) |
| MLV-HBSS + saline | 3.1 | 5/5 | 699 ± 322 | 5/5 | 89, >100, >100, >100, | 4.1 ± 0.8 |
| | | | | | >100 | |
| MLV-HBSS + CPT-11 | 1.2 | 5/5 | 334 ± 88 | 5/5 | 42, 42, 45, 56, 79 | 2.6 ± 0.3 |
| MLV-JBT3002 (1.0 μg/dose) + CPT-11 | 1.3 | 5/5 | 157 ± 96 | 4/5 | 0, 1, 9, 11, 13 | 1.5 ± 0.2 |
| FF-JBT3002 (1.0 μg/dose) + CPT-11 | −1.4 | 5/5 | 235 ± 78 | 5/5 | 34, 41, 56, 70, 88 | 2.6 ± 0.6 |
| FF-JBT3002 (0.1 μg/dose) + CPT-11 | −0.2 | 5/5 | 189 ± 13 | 5/5 | 3, 12, 16, 24, 34 | 1.6 ± 0.4 |
| FF-JBT3002 (0.01 μg/dose) + CPT-11 | 0.3 | 5/5 | 214 ± 45 | 5/5 | 2, 4, 13, 31, 40 | 1.6 ± 0.3 |
| FF-JBT3002 (0.001 μg/dose) + CPT-11 | 2.5 | 5/5 | 237 ± 20 | 5/5 | 31, 42, 47, 58, 69 | 2.8 ± 0.7 |
| FF-JBT3002 (0.0001 μg/dose) + CPT-11 | 2.3 | 5/5 | 225 ± 34 | 5/5 | 30, 32, 48, 52, 83 | 2.7 ± 0.9 |

BALB/c mice were injected into the spleen with 1 × 10⁴ viable CT-26 cells on day 0. Groups of mice were treated with oral feedings of 5 μmol
MLV-HBSS, MLV-JBT3002 (1 μg/dose), or FF-JBT3002 (at either 1.0, 0.1, 0.001, or 0.0001 μg/dose) thrice weekly for 3 weeks beginning 3 days after
tumor cell inoculation. Some mice received an i.p. injection of 100 mg/kg CPT-11 once a week (on days 7, 14, and 21). All groups were killed on day
23.

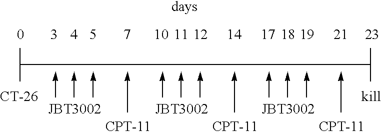

^{a}Changes in body weight were calculated by the formula; ΔBW (%) = (A − B)B × 100, where A = mean body weight of mice at death, and B = mean
body weight of mice on day 0.

TABLE 14
Therapy of experimental liver metastasis produced by murine CT-26 colon carcinomas with intensive CPT-11
injections in combination with either MLV-JBT 3002 or free-form (FF) JBT-3002 at different doses

| | | | Spleen tumor | | Liver metastasis | | |
|---|---|---|---|---|---|---|---|
| | ΔBW^{a} | ΔBW₂₁^{a} | | Mean tumor | | | Liver weight |
| Treatment | (%) | (%) | Incidence | volume (mm³) | Incidence | No | (g) |
| Control | 2.9 | 6.9 | 5/5 | 353 ± 29 | 5/5 | 54, >100, >100, >100, >100 | 3.4 ± 1.1 |
| CPT-11 | −24.0 | ND | 5/5^{b} | 35 ± 16 | 0/5^{b} | all 0 | 1.2 ± 0.2 |
| MLV-JBT 3002 (1.0 μg/dose) + CPT-11 | −9.4 | −7.6 | 5/5 | 75 ± 64 | 3/5 | 0, 0, 3, 5, 16 | 1.5 ± 0.1 |
| FF-JBT 3002 (0.05 μg/dose) + CPT-11 | −6.8 | −6.0 | 5/5 | 83 ± 70 | 4/5 | 0, 1, 9, 18, 21 | 1.7 ± 0.0 |

BALB/c mice were injected into the spleen with 1 × 10⁴ viable CT-26 cells on day 0. Mice were treated with oral feedings of 5 μmol MLV-JBT 3002 (1
μg/dose), or FF-JBT-3002 (0.05 μg/dose) for 3 consecutive days beginning 3 days after tumor cell inoculation. Seven days later, groups of mice received
4 daily i.p. injections of 100 mg/kg CPT-11. All groups were killed on day 21.

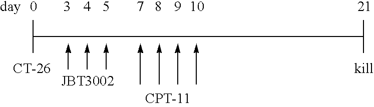

^{a}Changes in body weight were calculated by the formula: ΔBW (%) = (A − B)B × 100, where A = mean body weight of mice on the indicated day, and
B = mean body weight of mice on day 0.
^{b}All mice died during therapy (3 mice on day 15 and 2 mice on day 16).
ND, not determined.

TABLE 15
Therapy of experimental liver metastaswis produced by murine CT-26 colon carcinoma with intensive CPT-11
injections in combination with oral JBT 3002

| | Spleen tumor | | Liver metastasis | | | |
|---|---|---|---|---|---|---|
| | | Mean tumor | | | | Liver weight |
| Treatment | Incidence | volume (mm³) | Incidence | No. | P^{a} | (g) |
| Control | 10/10 | 594 ± 51 | 10/10 | 85, >100, >100, >100, >100 | | 3.2 ± 0.9 |
| | | | | >100, >100, >100, >100, >100 | | |
| CPT-11 | 6/10^{b} | 79 ± 38^{c,e} | 1/10^{b} | 0, 0, 0, 0, 0, 0, 0, 0, 0, 26 | <0.0001 | 1.9 ± 0.3^{c,d} |
| JBT 3002 | 10/10 | 88 ± 34^{f} | 9/10 | 0, 1, 2, 6, 10, 10, 11, 15, 22, 31 | <0.0001 | 1.6 ± 0.2^{e} |
| JBT 3002 + CPT-11 | 4/10 | 47 ± 26^{f} | 4/10 | 0, 0, 0, 0, 0, 0, 2, 5, 5, 8 | <0.001 | 1.4 ± 0.1^{f} |

BALB c mice were injected into the spleen with 1 × 10⁴ viable CT-26 cells on day 0. Groups of mice were treated with
oral feedings of JBT 3002 (0.05 μg/dose) for 3 consecutive days beginning 3 days after tumor cell inoculation. Seven
days later, some mice received 4 daily i.p. injections of 100 mg/kg CPT-11. All groups were killed on day 21.

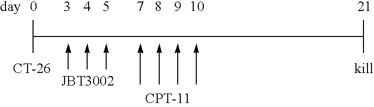

^{a}As compared with control.
^{b}Seven mice died during therapy (day 10, 13, 13, 14, 14, 17, 20).
^{c}Calculated from survive mice.
^{d}P < 0.05 as compared with control
^{e}P < 0.001 as compared with control
^{f}P < 0.0001 as compared with control.

TABLE 16
Therapy of experimental liver metastasis produced by murine CT-26 colon carcinoma with once weekly CPT-11
injections in combinations with oral JBT 3002

| | Spleen tumor | | Liver metastasis | | | |
|---|---|---|---|---|---|---|
| | | Mean tumor | | | | Liver weight |
| Treatment | Incidence | volume (mm³) | Incidence | No. | P^{a} | (g) |
| Control | 10/10 | 574 ± 101 | 10/10 | 72, >100, >100, >100, >100 | | 4.3 ± 1.0 |
| | | | | >100, >100, >100, >100, >100 | | |
| CPT-11 | 7/10 | 116 ± 32^{b} | 8/10 | 0, 0, 1, 5, 6, 13, 33, 81, 85, | 0.0005 | 2.0 ± 0.9^{c} |
| | | | | >100 | | |
| JBT 3002 | 8/10 | 241 ± 84 | 9/10 | 1, 2, 50, >100, >100, >100 | | 4.2 ± 1.6 |
| | | | | >100, >100, >100, >100, >100 | | |
| JBT 3002 + CPT-11 | 6/10 | 76 ± 34^{b} | 5/10 | 0, 0, 0, 0, 0, 1, 6, 37, 57 | <0.0001 | 1.7 ± 0.4^{c} |

BALB c mice were injected into the spleen with 1 × 10⁴ viable CT-26 cells on day 0. Groups of mice were treated with
oral feedings of JBT3002 (0.05 μg dose) thrice weekly for 3 weeks beginning 3 days after tumor cell inoculation.
Some mice received an i.p. injection of 100 mg/kg CPT-11 once a week (on days 7, 14, and 21). All groups were killed
on day 24.

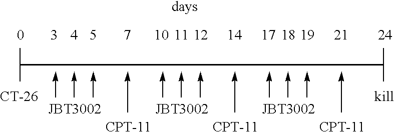

^{a}As compared with control
^{b}P < 0.05 as compared with control
^{c}P < 0.0001 as compared with control

TABLE 17
Induction of NO production in macrophages by free-form, formula 1, and
formula 2 JBT 3002

| | |
|---|---|
| 1. | Macrophages: TG-Mø from C57BL/6 mice. |
| 2. | Treatment of macrophages: Macrophages in 96-well plates (10⁵/ |
| | well were incubated for 24 hr with JBT in the presence or absence of |
| | IFN-γ (10 U/ml). Nitrite in the culture medium was then determined. |
| 3. | Results: |

| | | | Formula-1 JBT | | Formula 2-JBT | |
|---|---|---|---|---|---|---|
| JBT conc. | Free JBT | | (pH 1.5-7) | | (pH 8) | |
| (ng/ml) | medium | IFN-g | medium | IFN-g | medium | IFN-g |
| 10 | 8.4 | 60.9* | 2 | 50.7 | 2 | 47.4 |
| 2 | 0 | 53.1 | 0 | 38.6 | 0 | 38.1 |
| 0.4 | 0 | 44.7 | 0 | 34.8 | 0 | 33.5 |
| 0.08 | 0 | 41 | 0 | 25.5 | 0 | 20 |
| 0.016 | 0 | 33.7 | 0 | 6.3 | 0 | 1.9 |
| 0.003 | 0 | 17.5 | 0 | 0.4 | 0 | 0.7 |
| 0.0006 | n.d. | n.d. | 0 | 0.5 | 0 | 2 |
| 0 | 0 | 0.6 | | | | |

• nitrite: μM.

TABLE 18
Induction of NO production by JBT 3002.

| 1. | Materials and Methods | |
|---|---|---|
| 1) | Macrophages: C57BL/6 mice, TG-Mø, 10⁵ cells/well in 96-well plate. | |
| 2) | Treatment: with 10 U/ml of IFN-γ and various concentrations of | |
| | JBT3002 for 24 hr in 200 μl/well MEM-5% FBS. Nitrite | |
| | (100 μl/well) was measured. | |
| 2. | Results | <--------------TABLETS--------------> |

| JBT3002 | Free form | | filtered | | unfiltered | |
|---|---|---|---|---|---|---|
| (ng/ml) | Medium | IFN-γ | medium | IFN-γ | medium | IFN-γ |
| 10 | 0.5 | 47.1 | 0 | 41.0 | 7.0 | 53.0 |
| 1 | 0 | 37.7 | 0 | 29.3 | 0 | 44.5 |
| 0.1 | 0 | 27.7 | 0 | 20.9 | 0 | 34.1 |
| 0.01 | 0 | 19.5 | 0 | 7.7 | 0 | 26.2 |
| 0.001 | 0 | 8.5 | 0 | 0 | 0 | 4.3 |
| 0.0001 | 0 | 0 | 0 | 0 | n.d. | n.d. |
| 0 | 0 | 0 | | | | |

TABLE 19A
Tumor weight and incidence of metastases of L3.6pl human pancreatic tumors in nude mice after 4 weeks
treatment with 100 mg/kg CPT-11 i.p. once a week +/− oral feeding of JBT 3002 (tablet) 0.05 mcg/dose

| Treatment start with CPT11: 7 days after orthotopic tumor cell injection | | | | | | | |
|---|---|---|---|---|---|---|---|
| Treatment start with JBT3002: 3 days after orthotopic tumor cell injection | | | | | | | |
| Treatment schedule: | wed | thurs | fri | sat | sun | mon | tues |
| | JBT3002 | JBT3002 | JBT3002 | — | — | CPT11 | — |

(animals were sacrified 31 days after tumor cell injection)

| | CPT11 | Incidence | | | CPT11 + JBT3002 | Incidence | | |
|---|---|---|---|---|---|---|---|---|
| animal | Tumor weight (mg) | liver met | LN met | WT/PC | Tumor weight (mg) | liver met | LN met | WT/PC |
| 1 | 80 | − | ++ | − | 60 | − | − | − |
| 2 | 375 | − | ++ | − | 201 | − | − | − |
| 3 | 241 | − | ++ | − | 208 | − | − | − |
| 4 | 0 | − | − | − | 78 | − | − | − |
| 5 | 98 | − | + | − | 365 | − | ++ | − |
| 6 | 0 | − | − | − | 0 | − | − | − |
| 7 | 318 | − | ++ | − | 118 | − | − | − |
| 8 | 137 | − | ++ | − | 175 | − | − | − |
| 9 | 205 | − | ++ | − | 199 | − | − | − |
| 10 | 67 | − | − | − | 140 | − | − | − |
| Median | 117.5 | 0/10 | 7/10 | 0/10 | 157.5 | 0/10 | 1/10 | 0/10 |
| Max | 375 | | | | 365 | | | |
| Min | 0 | | | | 0 | | | |
| Average | 152.10 | | | | 154.40 | | | |
| St.Dev. | 106.12 | | | | 75.20 | | | |

TABLE 19B
Tumor weight and incidence of metastses of L3.6pl human pancreatic tumors in nude mice after 4 weeks
treatment with 100 mg/kg CPT-11 i.p. once a week +/− oral feeding of JBT 3002 (tablet) 0.05 mcg/dose

Treatment start with CPT11: 7 days after orthotopic tumor cell injection
Treatment start with JBT3002: 3 days after orthotopic tumor cell injection

| Treatment schedule: | wed | thurs | fri | sat | sun | mon | tues |
|---|---|---|---|---|---|---|---|
| | JBT3002 | JBT3002 | JBT3002 | — | — | CPT11 | — |

(animals were sacrificed 31 days after tumor cell injection)

| | Control (HBSS) | Incidence | | | JBT-3002 | Incidence | | |
|---|---|---|---|---|---|---|---|---|
| animal | Tumor weight (mg) | liver met | LN met | WT./PC | Tumor weight (mg) | liver met | LN met | WT/PC |
| 1 | 534 | — | ++ | — | 862 | — | ++ | WT |
| 2 | 556 | — | ++ | WT/PC | 871 | — | + | — |
| 3 | 483 | — | ++ | — | 981 | + (5) | ++ | WT |
| 4 | 831 | + (1) | ++ | — | 621 | — | ++ | WT |
| 5 | 955 | + (1) | + | — | 362 | — | + | — |
| 6 | 73 | + (1) | ++ | — | 733 | — | ++ | — |
| 7 | 578 | — | ++ | — | 559 | — | — | — |
| 8 | 723 | ++ (1) | ++ | — | 820 | + (1) | + | − |
| 9 | 701 | — | ++ | WT | 547 | — | — | — |
| 10 | | — | ++ | WT | | — | — | — |
| Median | 578 | 4/10 | 10/10 | 3/10 | 733 | 2/9 | 7/10 | 3/10 |
| Max | 955 | | | | 981 | | | |
| Min | 73 | | | | 362 | | | |
| Average | 603.78 | | | | 706.22 | | | |
| St. Dev. | 176.64 | | | | 163.53 | | | |

TABLE 19C
Tumor weight and incidence of metastases of L3.6pl human pancreatic tumors in nude mice
after 4 weeks treatment with 100 mg/kg CPT-11 i.p. once a week +/− oral feeding of JBT
3002 (tablet) 0.05 mdg/dose

Treatment start with CPT11: 7 days after orthotopic tumor cell injection
Treatment start with JBT3002: 3 days after orthotopic tumor cell injection

| | | | | | | | |
|---|---|---|---|---|---|---|---|
| Treatment schedule: | wed | thurs | fri | sat | sun | mon | tues |
| | JBT3002 | JBT3002 | JBT3002 | — | — | CPT11 | — |

(animals were sacrified 31 days after tumor cell injection)

| | tumor weight in mg | | Incidence | |
|---|---|---|---|---|
| therapy | median (range) | | liver met. | LN met. |
| Control (HBSS) | 578 | (73-955) | 4/10 | 10/10 |
| JBT3002 | 733 | (362-981) | 2/9 | 7/10 |
| CPT11 | 117.5 | (0-375) | 0/10 | 7/10 |
| CPT11 + JBT3002 | 157.5 | (0-365) | 0/10 | 1/10 |

TABLE 20
Therapy of experimental liver 7 metasrasis produced by KM12SM human colon carcinoma with CPT-11 i.p. plus
oral JBT 3002 in nude mice

| | 7/20 | 7/27 | | | | | | | | | | | | | | | | | | | | |
|---|---|---|---|---|---|---|---|---|---|---|---|---|---|---|---|---|---|---|---|---|---|---|
| Intensive | M | M | T | W | R | F | S | S | M | T | W | R | F | S | S | M | T | W | R | F | S | S |
| #5594 | T | | | | | | | | | | | | | | | | | | | | | |
| #5595 | | | | | | | | | | | | | | | | | | | | | | |
| #5596 | T | | | | | C | C | C | C | | | | | | | | | | | | | |
| #5597 | | | | | | | | | | | | | | | | | | | | | | |
| #5598 | T | J | J | J | | | | | | | | | | | | | | | | | | |
| #5599 | | | | | | | | | | | | | | | | | | | | | | |
| #5600 | T | J | J | J | | C | C | C | C | | | | | | | | | | | | | |
| #5601 | | | | | | | | | | | | | | | | | | | | | | |
| Once a week | M | M | T | W | R | F | S | S | M | T | W | R | F | S | S | M | T | W | R | F | S | S |
| #5602 | T | | | | | | | | | | | | | | | | | | | | | |
| #5603 | | | | | | | | | | | | | | | | | | | | | | |
| #5604 | T | | | | | C (75) | | | | | | | C 5604: 75 | | | | | | | C | | |
| #5605 | | | | | | | | | | | | | 5605: 50 | | | | | | | | | |
| #5606 | T | J | J | J | | | | | J | J | J | | | | | J | J | J | | | | |
| #5607 | | | | | | | | | | | | | | | | | | | | | | |
| #5608 | T | J | J | J | | C(75) | | | J | J | | J | C 5608: 75 | | | J | J | J | | C | | |
| #5609 | | | | | | | | | | | | | 5609: 50 | | | | | | | | | |

T: KM12sm 1 + 10\hat{ }6 i.spl
J: FF-JBT3002 (0.05 mcg/dose) oral
C: CPT-11 (50 mg/kg) i.p.
H. SHINOHAPA July 22, 1998

TABLE 21
Therapy of experimental liver metastases produced by CT-26 murine colon carcinoma with CPT-11 i.p. plus oral
JBT 3002 (free-form or tablet) in BALB/c mice

| | | | 8/14 | | | | | | | | | | | | | | | | | | | | | | | | |
|---|---|---|---|---|---|---|---|---|---|---|---|---|---|---|---|---|---|---|---|---|---|---|---|---|---|---|---|
| | | | 0 | | | | | | | 7 | | | | | | | 14 | | | | | | | 21 | | | |
| | | | F | S | S | M | T | W | R | F | S | S | M | T | W | R | F | S | S | M | T | W | R | F | S | S | M |
| INTENSIVE | | | | | | | | | | | | | | | | | | | | | | | | | | | |
| TREATMENT | | | | | | | | | | | | | | | | | | | | | | | | | | | |
| Group | I | 7332 | T | | | | | | | | | | | | | | | | | | | | | | | | |
| | (n = 5) | | | | | | | | | | | | | | | | | | | | | | | | | | |
| | Control | | | | | | | | | | | | | | | | | | | | | | | | | | |
| | II | 7333 | T | | | | | | | C | C | C | C | | | | | | | | | | | | | | |
| | (n = 5) | | | | | | | | | | | | | | | | | | | | | | | | | | |
| | CPT-11 | | | | | | | | | | | | | | | | | | | | | | | | | | |
| | III | 7334 | T | | | J | J | J | | | | | | | | | | | | | | | | | | | |
| | (n = 5) | | | | | | | | | | | | | | | | | | | | | | | | | | |
| | FF-JBT | | | | | | | | | | | | | | | | | | | | | | | | | | |
| | IV | 7335 | T | | | J | J | J | | | | | | | | | | | | | | | | | | | |
| | (n = 5) | | | | | | | | | | | | | | | | | | | | | | | | | | |
| | TAB-JBT | | | | | | | | | | | | | | | | | | | | | | | | | | |
| | V | 7336 | T | | | J | J | J | C | C | C | C | | | | | | | | | | | | | | | |
| | (n = 5) | | | | | | | | | | | | | | | | | | | | | | | | | | |
| | FF-JBT/ | | | | | | | | | | | | | | | | | | | | | | | | | | |
| | CPT-11 | | | | | | | | | | | | | | | | | | | | | | | | | | |
| | VI | 7337 | T | | | J | J | J | C | C | C | C | | | | | | | | | | | | | | | |
| | (n = 5) | | | | | | | | | | | | | | | | | | | | | | | | | | |
| | TAB-JBT/ | | | | | | | | | | | | | | | | | | | | | | | | | | |
| | CPT-11 | | | | | | | | | | | | | | | | | | | | | | | | | | |
| ONCE A WEEK | | | | | | | | | | | | | | | | | | | | | | | | | | | |
| TREATMENT | | | | | | | | | | | | | | | | | | | | | | | | | | | |
| Group | I | 7338 | T | | | | | | | | | | | | | | | | | | | | | | | | |
| | (n = 5) | | | | | | | | | | | | | | | | | | | | | | | | | | |
| | Control | | | | | | | | | | | | | | | | | | | | | | | | | | |
| | II | 7339 | T | | | | | | | C | | | | | | | C | | | | | | | C | | | |
| | (n = 5) | | | | | | | | | | | | | | | | | | | | | | | | | | |
| | CPT-11 | | | | | | | | | | | | | | | | | | | | | | | | | | |
| | III | 7340 | T | | | J | J | J | | | | | J | J | J | | | | | J | J | J | | | | | |
| | (n = 5) | | | | | | | | | | | | | | | | | | | | | | | | | | |
| | FF-JBT | | | | | | | | | | | | | | | | | | | | | | | | | | |
| | IV | 7341 | T | | | J | J | J | | | | | J | J | J | | | | | J | J | J | | | | | |
| | (n = 5) | | | | | | | | | | | | | | | | | | | | | | | | | | |
| | TAB-JBT | | | | | | | | | | | | | | | | | | | | | | | | | | |
| | V | 7342 | T | | | J | J | J | | C | | | J | J | J | | C | | | J | J | J | | C | | | |
| | (n = 5) | | | | | | | | | | | | | | | | | | | | | | | | | | |
| | FF-JBT/ | | | | | | | | | | | | | | | | | | | | | | | | | | |
| | CPT-11 | | | | | | | | | | | | | | | | | | | | | | | | | | |
| | VI | 7343 | T | | | J | J | J | | C | | | J | J | J | | C | | | J | J | J | | C | | | |
| | (n = 5) | | | | | | | | | | | | | | | | | | | | | | | | | | |
| | TAB-JBT/ | | | | | | | | | | | | | | | | | | | | | | | | | | |
| | CPT-11 | | | | | | | | | | | | | | | | | | | | | | | | | | |

Legend
T: CT26, 10,000 cells, i.spl (by Shinohara and Ozawa)
C: CPT-11, 100 mg/kg, i.p. (by Shinohara and Ozawa)
J: JBT 3002 (free form or tablet solution), 0.05 mcg/dose, oral (by Jerry)
H. SHINAHARA Aug. 6, 1998

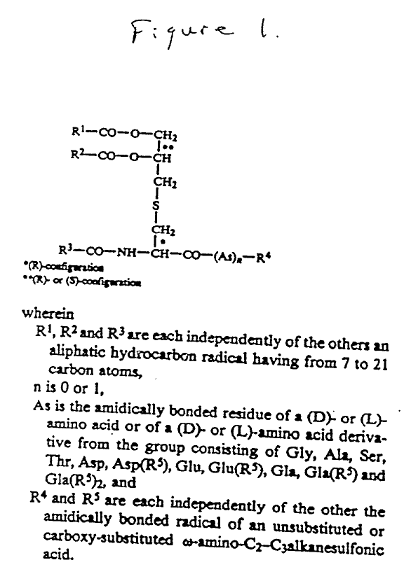

1. An isolated lipopeptide comprising the formula represented in FIG. 1.

2. The lipopeptide of claim 1, comprising a multilamellar liposome.

3. The lipopeptide of claim 1, comprising the formula represented in any one of FIG. 1 or FIG. 2.

4. The lipopeptide of claim 3, comprising a multilamellar liposome.

5. The lipopeptide of claim 1, comprising the formula represented in FIG. 2.

6. The lipopeptide of claim 5, further comprising a multilamellar liposome.

7. A pharmaceutical composition useful in the treatment of neoplasia, comprising: a therapeutically effective amount of the lipopeptide of claim 1; and a pharmaceutically acceptable carrier.

8. A pharmaceutical composition useful in the treatment of neoplasia, comprising: a therapeutically effective amount of the lipopeptide of claim 3; and a pharmaceutically acceptable carrier.

9. A pharmaceutical composition useful in the treatment of neoplasia, comprising: a therapeutically effective amount of the lipopeptide of claim 5; and a pharmaceutically acceptable carrier.

10. A pharmaceutical composition useful in the treatment of neoplasia, comprising: a therapeutically effective amount of the lipopeptide of claim 6; and a pharmaceutically acceptable carrier.

11. A pharmaceutical composition useful in the treatment of neoplasia, comprising: a first anti-neoplastic agent comprising a therapeutically effective amount of the lipopeptide of claim 1; a multilamellar liposome; a therapeutically effective amount of a second anti-neoplastic agent; and a pharmaceutically acceptable carrier.

12. A pharmaceutical composition useful in the treatment of neoplasia, comprising: a first anti-neoplastic agent comprising a therapeutically effective amount of the lipopeptide of claim 3; a multilamellar liposome; a therapeutically effective amount of a second anti-neoplastic agent; and a pharmaceutically acceptable carrier.

13. A pharmaceutical composition useful in the treatment of neoplasia, comprising: a first anti-neoplastic agent comprising a therapeutically effective amount of the lipopeptide of claim 5; a therapeutically effective amount of a second anti-neoplastic agent; and a pharmaceutically acceptable carrier.

14. A pharmaceutical composition useful in the treatment of neoplasia, comprising: a first anti-neoplastic agent comprising a therapeutically effective amount of the lipopeptide of claim 6; a therapeutically effective amount of a second anti-neoplastic agent; and a pharmaceutically acceptable carrier.

15. The pharmaceutical composition of claim 13, wherein said second anti-neoplastic agent or therapeutic is selected from the group consisting of: CPT-11; topoisomerase I inhibitors; paclitaxel; taxotere; modified taxane analogs; cisplatin; doxorubicin; and ifosfamide.

16. The pharmaceutical composition of claim 14, wherein said second anti-neoplastic agent or therapeutic is selected from the group consisting of: CPT-11; topoisomerase I inhibitors; paclitaxel; taxotere; modified taxane analogs; cisplatin; doxorubicin; and ifosfamide.

17. A pharmaceutical composition useful in the treatment of neoplasia, comprising: a therapeutically effective amount of the lipopeptide of claim 1; a multilamellar liposome; a therapeutically effective amount of one or more cytokines; and a pharmaceutically acceptable carrier.

18. A pharmaceutical composition useful in the treatment of neoplasia, comprising: a therapeutically effective amount of the lipopeptide of claim 4; a multilamellar liposome; a therapeutically effective amount of one or more cytokines; and a pharmaceutically acceptable carrier.

19. A pharmaceutical composition useful in the treatment of neoplasia, comprising: a therapeutically effective amount of the lipopeptide of claim 5; a therapeutically effective amount of one or more cytokines; and a pharmaceutically acceptable carrier.

20. A pharmaceutical composition useful in the treatment of neoplasia, comprising: a therapeutically effective amount of the lipopeptide of claim 6; a therapeutically effective amount of one or more cytokines; and a pharmaceutically acceptable carrier.

21. The pharmaceutical composition of claim 19, wherein said one or more cytokines is selected from the group consisting of: TNF-α; IL-1β; IL-6; G-CSF; GM-CSF.

22. The pharmaceutical composition of claim 20, wherein said one or more cytokines is selected from the group consisting of: TNF-α; IL-1β; IL-6; G-CSF; GM-CSF.

23. A method of treating neoplasia, comprising: administering to a subject with neoplasia by a clinically acceptable route of delivery a therapeutically effective amount of the pharmaceutical composition of claim 7.

24. A method of treating neoplasia, comprising: administering to a subject with neoplasia by a clinically acceptable route of delivery a therapeutically effective amount of the pharmaceutical composition of claim 8.

25. A method of treating neoplasia, comprising: administering to a subject with neoplasia by a clinically acceptable route of delivery a therapeutically effective amount of the pharmaceutical composition of claim 9.

26. A method of treating neoplasia, comprising: administering to a subject with neoplasia by a clinically acceptable route of delivery a therapeutically effective amount of the pharmaceutical composition of claim 10.

27. A method of treating neoplasia, comprising: administering to a subject with neoplasia by a clinically acceptable route of delivery a therapeutically effective amount of the pharmaceutical composition of claim 11.

28. A method of treating neoplasia, comprising: administering to a subject with neoplasia by a clinically acceptable route of delivery a therapeutically effective amount of the pharmaceutical composition of claim 12.

29. A method of treating neoplasia, comprising: administering to a subject with neoplasia by a clinically acceptable route of delivery a therapeutically effective amount of the pharmaceutical composition of claim 13.

30. A method of treating neoplasia, comprising: administering to a subject with neoplasia by a clinically acceptable route of delivery a therapeutically effective amount of the pharmaceutical composition of claim 14.

31. A method of treating neoplasia, comprising: administering to a subject with neoplasia by a clinically acceptable route of delivery a therapeutically effective amount of the pharmaceutical composition of claim 15.

32. A method of treating neoplasia, comprising: administering to a subject with neoplasia by a clinically acceptable route of delivery a therapeutically effective amount of the pharmaceutical composition of claim 16.

33. A pharmaceutical composition useful in the treatment of a side effect resulting from treatment of a subject with neoplasia, comprising: a therapeutically effective amount of the lipopeptide of claim 5; and a pharmaceutically acceptable carrier.

34. A pharmaceutical composition useful in the treatment of a side effect resulting from treatment of a subject with neoplasia, comprising: a therapeutically effective amount of the lipopeptide of claim 6; and a pharmaceutically acceptable carrier.

35. A method of treating a subject being treated with a neoplastic agent or therapeutic in an amount sufficient to cause a side effect, which method comprises administering to said subject the pharmaceutical composition of claim 33, in an amount effective to alleviate or prevent said side effect.

36. A method of treating a subject being treated with a neoplastic agent or therapeutic in an amount sufficient to cause a side effect selected from the group consisting of myelosupression, mucositis, and peripheral neuropathy, which method comprises administering to said subject the pharmaceutical composition of claim 33, in an amount effective to alleviate or prevent said side effect.

37. A method of treating a subject being treated with a neoplastic agent or therapeutic in an amount sufficient to cause a side effect, which method comprises administering to said subject the pharmaceutical composition of claim 34, in an amount effective to alleviate or prevent said side effect.

38. A method of treating a subject being treated with a neoplastic agent or therapeutic in an amount sufficient to cause a side effect selected from the group consisting of: myelosupression, mucositis, and peripheral neuropathy, which method comprises administering to said subject the pharmaceutical composition of claim 34, in an amount effective to alleviate or prevent said side effect.

39. A method of treating neoplasia, comprising: administering to a subject with neoplasia by a clinically acceptable route of delivery a therapeutically effective amount of the pharmaceutical composition of claim 17.

40. A method of treating neoplasia, comprising: administering to a subject with neoplasia by a clinically acceptable route of delivery a therapeutically effective amount of the pharmaceutical composition of claim 18.

41. A method of treating neoplasia, comprising: administering to a subject with neoplasia by a clinically acceptable route of delivery a therapeutically effective amount of the pharmaceutical composition of claim 19.

42. A method of treating neoplasia, comprising: administering to a subject with neoplasia by a clinically acceptable route of delivery a therapeutically effective amount of the pharmaceutical composition of claim 20.

43. A method of treating neoplasia, comprising: administering to a subject with neoplasia by a clinically acceptable route of delivery a therapeutically effective amount of the pharmaceutical composition of claim 21.

44. A method of treating neoplasia, comprising: administering to a subject with neoplasia by a clinically acceptable route of delivery a therapeutically effective amount of the pharmaceutical composition of claim 22.

45. A pharmaceutical composition useful in the treatment of neoplasia, comprising: a first anti-neoplastic agent comprising a therapeutically effective amount of a lipopeptide; a therapeutically effective amount of a second anti-neoplastic agent; and a pharmaceutically acceptable carrier, wherein the first neoplastic agent comprises a lipopeptide selected from the group consisting of: MTP-PE; MLV-MTP-PE; CGP31362; MLV-CGP31362; JBT3002; and MLV-JBT3002.

46. A method of treating neoplasia, comprising: administering to a subject with neoplasia by a clinically acceptable route of delivery a therapeutically effective amount of the pharmaceutical composition of claim 45.

47. The pharmaceutical composition of claim 7, further comprising a pharmaceutically acceptable carrier in tablet form.

48. The pharmaceutical composition of claim 8, further comprising a pharmaceutically acceptable carrier in tablet form.

49. A method of upregulating IL-15 production comprising, administering to a subject a pharmaceutical composition that comprises an isolated lipopeptide comprising the formula represented in FIG. 1.

50. A method of upregulating IL-15 production comprising, administering to a subject a pharmaceutical composition that comprises an isolated lipopeptide comprising the formula represented in FIG. 2.

51. A method of treating a subject being treated with a neoplastic agent or therapeutic in an amount sufficient to cause a side effect, which method comprises administering to said subject a pharmaceutical composition that in a therapeutically effective concentration upregulates IL-15 production.

52. The method of claim 51, wherein said pharmaceutical composition comprises an isolated lipopeptide comprising the formula represented in FIG. 1.

53. The method of claim 51, wherein said pharmaceutical composition comprises an isolated lipopeptide comprising the formula represented in FIG. 2.
